(19) United States
(12) Patent Application Publication
(10) Pub. No.: US 2026/0279507 A1
(43) Pub. Date: Sep. 17, 2026

(54) Recognition-Enhanced Material Design System: Methods and Computational Systems for Predicting and Optimizing Novel Materials Using Recognition Science

(71) Applicant: Jonathan Washburn, Austin, TX (US)

(72) Inventor: Jonathan Washburn, Austin, TX (US)

(21) Appl. No.: 19/564,011

(22) Filed: Mar. 11, 2026

Related U.S. Application Data
(60) Provisional application No. 63/771,436, filed on Mar. 13, 2025.

(51) Int. Cl. G16C 20/20

(52) U.S. Cl. CPC G16C 20/20

(57) ABSTRACT

A recognition-enhanced material design system is disclosed for evaluating, predicting, screening, ranking, and optimizing candidate materials using a recognition-based computational framework. Candidate-material inputs are received and processed to generate one or more recognition-compatible metrics. A recognition coverage function is applied to at least one metric to produce recognition-weighted outputs, including structure-, property-, composition-, and stability-related outputs. In some embodiments, outputs from multiple subsystems are combined to determine an overall material score and to generate ranking, screening, prediction, optimization, or prioritization results. The framework can interface with external computational platforms, including first-principles simulation, molecular dynamics, machine-learning, and database platforms, to support candidate-material discovery across multiple material classes.

### CROSS-REFERENCE TO RELATED APPLICATIONS

This application claims the benefit of U.S. Provisional Patent Application No. 63/771,436, filed on Mar. 13, 2025, titled “Recognition-Enhanced Material Design System: Methods and Computational Systems for Predicting and Optimizing Novel Materials Using Recognition Physics,” the entire contents of which are hereby incorporated by reference herein.

### BACKGROUND OF THE INVENTION

The present disclosure relates generally to computational materials science, materials informatics, and computer-implemented materials discovery, and more particularly to computational evaluation environments in which candidate materials are analyzed with respect to structural, electronic, thermodynamic, chemical, mechanical, optical, magnetic, transport, or electrochemical characteristics.

Computational materials discovery commonly relies on simulation, modeling, and database-driven evaluation to identify candidate materials having desired combinations of properties. In many practical workflows, large numbers of candidate materials are generated or selected, and each candidate is then subjected to one or more computational analyses to estimate structural, thermodynamic, electronic, mechanical, optical, or transport-related behavior.

Density functional theory is widely used for first-principles evaluation of material systems because it can provide atomistic and electronic-structure information for crystalline and non-crystalline materials. However, density functional theory calculations can be computationally expensive, particularly when screening large candidate sets, exploring large unit cells, treating defects or interfaces, or using higher-accuracy functionals. As a result, exhaustive evaluation of broad materials spaces can require substantial computing resources and long turnaround times. Molecular dynamics techniques can provide favorable scaling for many atomistic simulations and can be useful for evaluating structural evolution, temperature-dependent behavior, and dynamic effects. However, such techniques often depend on force fields or interaction models that are tailored to particular chemical families, processing conditions, or structural regimes. This can limit transferability across broader material classes or across candidate spaces that differ materially from the systems used during force-field development.

Machine-learning-based materials models and machine-learning interatomic potentials can improve throughput in some settings, but such models commonly depend on substantial labeled datasets, architecture choices, training procedures, and distributional assumptions. In practice, predictive performance may degrade when candidate materials depart from the domain represented in the training data, thereby limiting robustness in exploratory or out-of-distribution discovery workflows. Conventional materials-screening pipelines also tend to rely on separate evaluation logic for different tasks. For example, one workflow may use one metric for structure relaxation, another metric for property scoring, another metric for composition ranking, and still another metric for thermodynamic filtering. This fragmentation can make it difficult to apply a unified prioritization approach across different stages of candidate evaluation.

In many computational screening environments, instability filtering is performed using hard thresholds or binary exclusion rules. While binary filtering can remove clearly unsuitable candidates, such approaches may fail to provide an informative graded ranking across candidates that are near a threshold or that differ meaningfully in relative stability margin.

In many composition-design settings, candidate spaces are combinatorially large. This is particularly true for multicomponent alloys, doped materials, substituted crystal families, layered materials, and broad search spaces involving multiple elemental choices and stoichiometric possibilities. Without an efficient prioritization framework, downstream evaluation cost can increase rapidly.

In addition, many practical materials-design environments involve integration of multiple external tools, such as first-principles engines, molecular dynamics engines, machine-learning predictors, and database interfaces. Existing workflows may therefore benefit from computational approaches that can operate with such tools in a compatible manner without requiring full redevelopment of the underlying external engines.

Accordingly, a need remains for improved computational systems and methods for screening, ranking, predicting, and optimizing candidate materials in a manner that is computationally efficient, broadly applicable across material classes, compatible with existing computational workflows, and capable of producing useful prioritization outputs for further analysis and validation.

### SUMMARY OF THE INVENTION

The present disclosure provides computational systems, methods, apparatuses, and non-transitory computer-readable media for evaluating, predicting, screening, ranking, and optimizing candidate materials using a recognition-enhanced computational framework applied to materials representations, simulation outputs, predictive-model outputs, database-derived information, experimentally derived information, or combinations thereof. In some embodiments, the disclosed framework operates as a post-processing and reweighting layer that interfaces with one or more existing computational chemistry, materials simulation, or materials informatics environments without requiring re-implementation of the underlying engines.

In some embodiments, the disclosed framework applies a recognition coverage function denoted Fcov(r) and defined as Fcov(r)=r/(r+Xopt), where r is a nonnegative input metric and Xopt is a positive recognition constant. In some embodiments, Xopt=phi/pi, where phi=(1+sqrt(5))/2. In some embodiments, the recognition coverage function increases as r increases, equals zero when r equals zero, and remains less than one for finite nonnegative values of r, thereby providing a continuous weighting mechanism that can be used across multiple materials-design tasks. In some embodiments, the disclosed framework comprises a plurality of cooperating recognition-enhanced subsystems. In some embodiments, such subsystems include a recognition-based structure optimization algorithm, a recognition-based property prediction algorithm, a recognition composition optimization subsystem, and a recognition stability screening subsystem. In some embodiments, the subsystems are used individually. In some embodiments, the subsystems are used in serial combination, parallel combination, iterative workflows, closed-loop discovery workflows, or other coordinated candidate-evaluation pipelines.

In some embodiments, the recognition-based structure optimization algorithm is configured to process structural information associated with a candidate material and to generate one or more recognition-weighted structural outputs. In some embodiments, the structural information includes atomic positions, lattice information, interatomic distances, pairwise interaction values, neighborhood relationships, graph relationships, force-related quantities, energy-related quantities, or combinations thereof. In some embodiments, the recognition-based structure optimization algorithm computes pairwise terms by applying the recognition coverage function to a distance-related metric and combining the resulting values with interaction metrics to produce a structure-related score, structure-related energy, structure-related ranking value, relaxed candidate representation, or other structure-related output. In some embodiments, longer-range interactions are weighted in a bounded and monotone manner without introducing additional recognition-specific force-field parameters.

In some embodiments, the recognition-based property prediction algorithm is configured to process one or more baseline property values associated with a candidate material and to generate one or more recognition-weighted property outputs. In some embodiments, a baseline property value is derived from density functional theory, molecular dynamics, a machine-learning model, a database, an empirical model, an experimentally derived quantity, or another computational or measurement source. In some embodiments, one or more property terms are computed by multiplying a property weighting factor, a baseline property metric, and a recognition coverage value derived from a recognition metric associated with that property. In some embodiments, the recognition-weighted property outputs are aggregated to produce a predicted property value, a ranking value, a screening value, a thresholding value, a prioritization output, or another property-related output. In some embodiments, the recognition weighting does not inflate the baseline property magnitude and instead provides a bounded reweighting useful for candidate screening and ranking.

In some embodiments, the recognition composition optimization subsystem is configured to process composition information associated with a candidate material and to generate one or more recognition-weighted composition outputs. In some embodiments, the composition information includes elemental identities, stoichiometric values, site occupations, dopant selections, substitution candidates, alloy fractions, or other composition-defining information. In some embodiments, a recognition-based composition weight for an element or other compositional constituent is determined by applying the recognition coverage function to a cohesive-energy-related metric or another composition-associated metric. In some embodiments, a composition score is determined by combining such recognition-based composition weights with corresponding composition fractions. In some embodiments, the composition optimization subsystem provides a parameter-light or parameter-free ranking of composition space using cohesive-energy information and can be used to navigate large composition search spaces before downstream simulation or validation.

In some embodiments, the recognition stability screening subsystem is configured to process one or more stability-related metrics associated with a candidate material and to generate one or more recognition-weighted stability outputs. In some embodiments, the stability-related metric includes a formation enthalpy metric, an energy-above-hull metric, a decomposition-energy metric, a free-energy metric, a phase-stability metric, or another thermodynamic or quasi-thermodynamic stability metric. In some embodiments, a stability score is computed by applying the recognition coverage function to a nonnegative stability-related metric. In some embodiments, the nonnegative stability-related metric is based on the absolute value of a formation enthalpy quantity. In some embodiments, the resulting stability score is used to rank candidates, filter candidates, compute a passing fraction, define a threshold policy, or reduce a candidate set prior to more computationally intensive downstream analysis. In some embodiments, the recognition stability screening subsystem replaces a binary stable-versus-unstable decision with a graded priority ranking.

In some embodiments, outputs from two or more recognition-enhanced subsystems are combined to generate an overall candidate-material score. In some embodiments, a combined score is determined by combining a property-related score, a stability-related score, and a composition-related score using corresponding weighting values. In some embodiments, the weighting values are user-selected, application-specific, or determined according to a parameter policy derived from Xopt. In some embodiments, the combined score is used to rank candidate materials for additional simulation, synthesis, experimental validation, qualification, or deployment.

In some embodiments, the disclosed framework is configured to reduce computational burden by deprioritizing lower-ranked or lower-stability candidates before expensive simulation stages are performed. In some embodiments, the disclosed framework is configured to reduce the size of an experimental validation set by selecting a smaller subset of candidates having higher recognition-weighted scores. In some embodiments, the disclosed framework is applied to metals, semiconductors, ceramics, polymers, two-dimensional materials, perovskites, metal-organic frameworks, battery materials, and other material classes while using the same recognition coverage function across multiple subsystems.

In some embodiments, the disclosed framework is integrated with one or more external computational platforms, including density functional theory platforms, molecular dynamics platforms, machine-learning materials platforms, database-driven screening environments, and hybrid computational workflows. In some embodiments, the disclosed framework is implemented as a local software package, a distributed computing workflow, a high-performance computing workflow, an application programming interface, a plugin, a cloud-executed service, or another computational deployment architecture. In some embodiments, the disclosed framework operates as a post-processing wrapper on existing density functional theory or molecular dynamics codes without modification of the underlying source code of those external platforms.

These and other features, embodiments, and advantages of the present disclosure will become more apparent from the following description, the drawings, and the claims.

### BRIEF DESCRIPTION OF THE DRAWINGS

The foregoing and other features of the present disclosure will become more fully apparent from the following description and appended claims, taken in conjunction with the accompanying drawings.

FIG. 1 is a schematic block diagram of a recognition-enhanced material design system configured to receive candidate-material inputs, process such inputs through a plurality of recognition-enhanced computational subsystems, and generate one or more ranking, screening, prediction, or optimization outputs.

FIG. 2 is a flow diagram illustrating a recognition-enhanced candidate-material evaluation workflow in which candidate materials are received, one or more recognition-based computational operations are performed, and one or more candidate materials are ranked, filtered, or selected for further analysis, simulation, or validation.

FIG. 3 is a graph illustrating a recognition coverage function used in certain embodiments of the present disclosure, wherein the recognition coverage function increases in a bounded manner as an input metric increases.

FIG. 4 is a flow diagram illustrating a recognition-based structure optimization process in which structural information associated with a candidate material is processed to generate one or more recognition-weighted structural outputs.

FIG. 5 is a schematic diagram illustrating an example pairwise interaction evaluation process in which interatomic distances and interaction-related values are combined under a recognition-enhanced framework to generate a structure-related output.

FIG. 6 is a flow diagram illustrating a recognition-based property prediction process in which one or more baseline property values are processed using one or more recognition metrics to generate one or more recognition-weighted property outputs.

FIG. 7 is a flow diagram illustrating a recognition composition optimization process in which composition information associated with a candidate material is processed to generate one or more recognition-weighted composition scores.

FIG. 8 is a flow diagram illustrating a recognition stability screening process in which one or more stability-related metrics associated with a candidate material are processed to generate one or more recognition-weighted stability outputs.

FIG. 9 is a flow diagram illustrating a combined candidate-material scoring process in which outputs from a plurality of recognition-enhanced computational subsystems are combined to generate an overall candidate-material score.

FIG. 10 is a schematic diagram illustrating an example computing environment in which the disclosed recognition-enhanced framework is implemented using one or more processors, one or more memory devices, and one or more local, distributed, or cloud-based computational resources.

### DETAILED DESCRIPTION OF THE INVENTION

Reference will now be made in detail to various embodiments of the present disclosure, examples of which are illustrated in the accompanying drawings. Wherever possible, the same reference numerals will be used throughout the drawings and the following description to refer to the same or like parts.

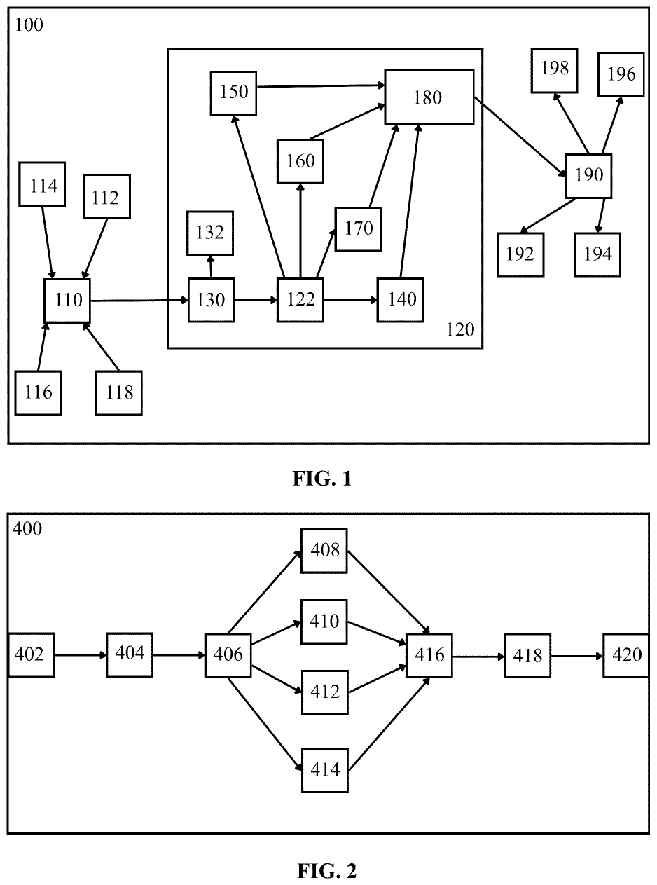

The present disclosure relates to a recognition-enhanced material design system 100 configured to evaluate, predict, rank, screen, and optimize candidate materials using a plurality of recognition-enhanced computational subsystems. In the embodiment illustrated in FIG. 1, the system 100 receives one or more candidate-material inputs 110 and processes the candidate-material inputs 110 through a recognition-enhanced computational framework 120 to generate one or more material-analysis outputs 190. In some embodiments, the material-analysis outputs 190 include ranking outputs, screening outputs, prediction outputs, optimization outputs, prioritization outputs, or combinations thereof.

In some embodiments, the candidate-material inputs 110 include one or more material representations 112, one or more simulation-derived inputs 114, one or more model-derived inputs 116, and one or more database-derived inputs 118. In some embodiments, the material representations 112 include atomic coordinates, lattice parameters, unit-cell definitions, symmetry data, bonding data, graph-based material representations, composition vectors, stoichiometric definitions, site-occupancy definitions, defect definitions, interface definitions, surface definitions, or combinations thereof. In some embodiments, the simulation-derived inputs 114 include outputs from density functional theory calculations, molecular dynamics calculations, interatomic-potential calculations, finite-element calculations, Monte Carlo calculations, or other computational materials analyses. In some embodiments, the model-derived inputs 116 include outputs from machine-learning models, surrogate models, regression models, classification models, or other predictive computational models. In some embodiments, the database-derived inputs 118 include values, descriptors, records, or candidate entries obtained from one or more materials databases, repositories, knowledge stores, or curated data sources.

In the embodiment of FIG. 1, the recognition-enhanced computational framework 120 includes a preprocessing and normalization module 130, a recognition-based structure optimization module 140, a recognition-based property prediction module 150, a recognition composition optimization module 160, a recognition stability screening module 170, and a combined scoring and ranking module 180. In some embodiments, the preprocessing and normalization module 130 receives the candidate-material inputs 110 and generates one or more normalized or otherwise transformed computational inputs for downstream processing. In some embodiments, the recognition-based structure optimization module 140, the recognition-based property prediction module 150, the recognition composition optimization module 160, and the recognition stability screening module 170 operate independently. In some embodiments, two or more of such modules operate in sequence, in parallel, iteratively, or in a closed-loop candidate-selection workflow.

In some embodiments, the preprocessing and normalization module 130 is configured to convert one or more raw material-related values into one or more recognition-compatible metrics. In some embodiments, such processing includes unit conversion, scaling, normalization, magnitude extraction, absolute-value conversion, clipping, threshold preparation, reference-value selection, dimensional-to-dimensionless conversion, or combinations thereof. In some embodiments, a distance-related quantity is normalized relative to a reference distance. In some embodiments, an energy-related quantity is normalized relative to a reference energy. In some embodiments, a property-related quantity is normalized relative to a reference property magnitude, a family-specific baseline, a material-class-specific baseline, or another selected reference quantity. In some embodiments, the recognition-enhanced computational framework 120 uses a recognition coverage function 122. In some embodiments, the recognition coverage function 122 is denoted Fcov(r) and defined as Fcov(r)=r/(r+Xopt), where r is a nonnegative input metric and Xopt is a positive recognition constant. In some embodiments, Xopt=phi/pi, where phi=(1+sqrt(5))/2. In some embodiments, the recognition coverage function 122 is monotone increasing with increasing r, equals zero when r equals zero, and remains less than one for finite nonnegative values of r. In some embodiments, the recognition coverage function 122 provides a bounded weighting mechanism that is applied across a plurality of different material-evaluation tasks within the system 100. In some embodiments, the preprocessing and normalization module 130 generates one or more recognition metrics 132 that are provided to one or more downstream modules. In some embodiments, the recognition metrics 132 include one or more normalized distance metrics, normalized interaction metrics, normalized property metrics, normalized composition metrics, normalized stability metrics, or combinations thereof. In some embodiments, the recognition metrics 132 are stored in one or more data structures in memory and are selectively routed to the recognition-based structure optimization module 140, the recognition-based property prediction module 150, the recognition composition optimization module 160, and the recognition stability screening module 170 depending on the selected workflow.

In some embodiments, the recognition-based structure optimization module 140 receives one or more structural inputs 142 and generates one or more structure-related outputs 148. In some embodiments, the structural inputs 142 include atomic positions, pairwise distance values, pairwise interaction values, local neighborhood definitions, bond-like descriptors, lattice descriptors, force-related quantities, energy-related quantities, or combinations thereof. In some embodiments, the recognition-based structure optimization module 140 computes one or more recognition-weighted pairwise values 144 by applying the recognition coverage function 122 to one or more distance-related metrics and combining the resulting recognition coverage values with one or more interaction-related quantities. In some embodiments, the recognition-weighted pairwise values 144 are aggregated to generate one or more structure-related outputs 148 including a structure score, a structure energy, a structure ranking value, a relaxed structure, or another structure-related computational output.

In some embodiments, the recognition-based property prediction module 150 receives one or more property inputs 152 and generates one or more property-related outputs 158. In some embodiments, the property inputs 152 include one or more baseline property values, one or more property descriptors, one or more model outputs, one or more simulation-derived values, or combinations thereof. In some embodiments, the recognition-based property prediction module 150 determines one or more recognition-weighted property terms 154 by combining a baseline property value with a corresponding recognition metric and, in some embodiments, a weighting factor. In some embodiments, the recognition-weighted property terms 154 are aggregated to generate one or more property-related outputs 158 including a predicted property value, a ranking value, a screening score, a thresholding value, a prioritization value, or another property-related output.

In some embodiments, the recognition composition optimization module 160 receives one or more composition inputs 162 and generates one or more composition-related outputs 168. In some embodiments, the composition inputs 162 include elemental identities, stoichiometric fractions, site assignments, substitution candidates, dopant selections, host compositions, alloy fractions, or combinations thereof. In some embodiments, the recognition composition optimization module 160 computes one or more recognition-weighted composition values 164 by applying the recognition coverage function 122 to one or more composition-associated metrics. In some embodiments, such composition-associated metrics include cohesive-energy-related values, binding-related values, site-preference-related values, compatibility-related values, or combinations thereof. In some embodiments, the recognition-weighted composition values 164 are combined to generate one or more composition-related outputs 168 including a composition score, a composition ranking value, a composition filter output, or a composition-prioritization output.

In some embodiments, the recognition stability screening module 170 receives one or more stability inputs 172 and generates one or more stability-related outputs 178. In some embodiments, the stability inputs 172 include one or more formation-enthalpy-related values, one or more energy-above-hull values, one or more decomposition-energy values, one or more free-energy values, one or more phase-stability values, one or more metastability values, or combinations thereof. In some embodiments, the recognition stability screening module 170 computes one or more recognition-weighted stability values 174 by applying the recognition coverage function 122 to one or more nonnegative stability-associated metrics. In some embodiments, the recognition-weighted stability values 174 are used to generate one or more stability-related outputs 178 including a stability score, a thresholding output, a filtered candidate subset, a pass-rate output, or a stability-based ranking output.

In some embodiments, the combined scoring and ranking module 180 receives outputs from two or more of the recognition-based structure optimization module 140, the recognition-based property prediction module 150, the recognition composition optimization module 160, and the recognition stability screening module 170 and generates one or more combined outputs. In some embodiments, the combined outputs include an overall material score, a ranked candidate list, a candidate-priority queue, a shortlist for further simulation, a shortlist for experimental validation, or another combined decision output. In some embodiments, the combined scoring and ranking module 180 combines a property-related score, a stability-related score, and a composition-related score according to a weighted combination policy. In some embodiments, the weighted combination policy uses fixed weights. In some embodiments, the weighted combination policy uses user-selected weights. In some embodiments, the weighted combination policy uses weights derived from Xopt.

In some embodiments, the material-analysis outputs 190 generated by the system 100 include one or more candidate rankings 192, one or more screening decisions 194, one or more prediction outputs 196, and one or more optimization outputs 198. In some embodiments, the candidate rankings 192 identify an ordering of candidate materials according to one or more recognition-weighted values. In some embodiments, the screening decisions 194 identify whether a candidate material is retained, deprioritized, rejected, flagged for further analysis, or passed to another stage of a computational workflow. In some embodiments, the prediction outputs 196 include one or more recognition-weighted estimates, scores, classifications, or property-related values. In some embodiments, the optimization outputs 198 include relaxed structures, updated compositions, refined candidate selections, adjusted workflow priorities, or other outputs resulting from operation of one or more of the modules of the framework 120.

In some embodiments, the system 100 interfaces with one or more external computational platforms 200, as shown for example in FIG. 10. In some embodiments, the external computational platforms 200 include one or more first-principles simulation platforms 210, one or more molecular dynamics platforms 220, one or more machine-learning platforms 230, one or more database platforms 240, and one or more workflow orchestration platforms 250. In some embodiments, the first-principles simulation platforms 210 include density functional theory engines. In some embodiments, the molecular dynamics platforms 220 include atomistic simulation engines configured to evaluate time-dependent or temperature-dependent behavior. In some embodiments, the machine-learning platforms 230 include models configured to predict one or more material properties, descriptors, or candidate rankings. In some embodiments, the database platforms 240 include local databases, remote databases, curated repositories, enterprise databases, or publicly accessible materials databases. In some embodiments, the workflow orchestration platforms 250 manage job submission, data transfer, candidate scheduling, caching, pipeline control, or combinations thereof.

In some embodiments, the system 100 communicates with the external computational platforms 200 through one or more interface layers 260. In some embodiments, the interface layers 260 include one or more file-based interfaces, one or more application programming interfaces, one or more plugin interfaces, one or more wrapper interfaces, one or more message-queue interfaces, or combinations thereof. In some embodiments, the interface layers 260 permit the system 100 to receive external outputs and provide downstream recognition-enhanced ranking, screening, prediction, or optimization operations without modifying the source code of the external computational platforms 200. In some embodiments, the system 100 stores or caches intermediate values received from the external computational platforms 200 so that subsequent recognition-enhanced analyses can be performed without repeating all prior external computations.

In some embodiments, the system 100 is implemented within a computing environment 300 shown in FIG. 10. In some embodiments, the computing environment 300 includes one or more processors 310, one or more memory devices 320, one or more storage devices 330, one or more communication interfaces 340, and one or more user interface components 350. In some embodiments, the one or more memory devices 320 store program instructions 322 which, when executed by the one or more processors 310, cause the one or more processors 310 to perform one or more of the operations described herein. In some embodiments, the one or more storage devices 330 store candidate-material records 332, recognition metrics 336, ranking results 338, and configuration data 339.

In some embodiments, the communication interfaces 340 permit communication between the computing environment 300 and one or more local systems, remote systems, cloud systems, cluster nodes, external computational platforms, or user devices. In some embodiments, the user interface components 350 include a display, a keyboard, a pointing device, a touchscreen, a command-line interface, a web interface, an application programming interface endpoint, or another user-accessible input or output mechanism. In some embodiments, the computing environment 300 is implemented as a local workstation. In some embodiments, the computing environment 300 is implemented as a distributed computing cluster. In some embodiments, the computing environment 300 is implemented in a cloud-computing environment. In some embodiments, the computing environment 300 is implemented in a hybrid architecture using local and remote computational resources.

In some embodiments, the disclosed technology is implemented as one or more non-transitory computer-readable media storing program instructions 322 which, when executed by the one or more processors 310, cause the one or more processors 310 to perform one or more of the candidate-material evaluation, recognition-metric generation, recognition coverage evaluation, recognition-weighted output generation, ranking, screening, prediction, optimization, prioritization, and combined scoring operations described herein. In some embodiments, the one or more non-transitory computer-readable media include one or more memory devices 320, one or more storage devices 330, or other machine-readable storage media that store executable instructions for implementing the preprocessing and normalization module 130, the recognition-based structure optimization module 140, the recognition-based property prediction module 150, the recognition composition optimization module 160, the recognition stability screening module 170, and the combined scoring and ranking module 180. In some embodiments, each such “module” comprises software instructions, routines, services, or tasks that are executed by the one or more processors 310 to perform the operations described herein.

In some embodiments, the system 100 operates on a candidate set including a plurality of candidate materials. In some embodiments, the candidate materials are generated by enumeration, retrieved from a database, generated by a machine-learning model, proposed by a user, derived from prior simulation outputs, or obtained from another source. In some embodiments, one or more candidates are processed through the preprocessing and normalization module 130 and then routed to one or more downstream modules based on a selected workflow. In some embodiments, all candidates are processed by all downstream modules. In some embodiments, a first subset of candidates is processed by the recognition stability screening module 170 before a second subset is passed to the recognition-based property prediction module 150 or the recognition-based structure optimization module 140. In some embodiments, the order of operations is varied based on computational cost, target property type, user preference, material class, or workflow policy.

In some embodiments, the disclosed system 100 provides a unified computational framework in which a common recognition coverage function 122 is used across a plurality of materials-analysis tasks. In some embodiments, this unified framework permits structural evaluation, property evaluation, composition evaluation, and stability evaluation to be performed under a common bounded weighting policy. In some embodiments, this common bounded weighting policy improves consistency across multiple stages of candidate analysis and provides a basis for comparing, filtering, and prioritizing candidate materials using outputs derived from different computational sources.

FIG. 2 illustrates an example recognition-enhanced candidate-material evaluation workflow 400 that may be carried out by the system 100. In some embodiments, the workflow 400 begins at step 402, where one or more candidate materials are received. In some embodiments, the candidate materials are received from a user, generated by a candidate-enumeration process, obtained from one or more databases, produced by one or more machine-learning systems, retrieved from prior simulation results, or obtained from another computational or experimental source.

In some embodiments, at step 404, the system 100 preprocesses the candidate materials and associated data using the preprocessing and normalization module 130. In some embodiments, preprocessing includes parsing structural definitions, reading composition information, importing baseline property values, importing stability-related values, selecting one or more reference quantities, converting units, normalizing one or more raw values, converting one or more signed values into one or more nonnegative metrics where appropriate, and preparing one or more recognition metrics 132 for downstream processing.

In some embodiments, at step 406, the system 100 selects one or more downstream recognition-enhanced evaluation paths for the candidate materials. In some embodiments, path selection is based on user configuration, material type, available input data, computational budget, target property type, workflow stage, or combinations thereof. In some embodiments, all candidate materials are routed through the same set of downstream modules. In some embodiments, different subsets of candidate materials are routed through different downstream modules.

In some embodiments, at step 408, one or more candidate materials are processed by the recognition stability screening module 170. In some embodiments, the recognition stability screening module 170 is used as an early-stage filter so that lower-priority or lower-stability candidates are removed or deprioritized before more computationally intensive downstream analysis is performed. In some embodiments, the recognition stability screening module 170 generates one or more stability-related outputs 178, including one or more stability scores, thresholding decisions, pass-rate values, or ranked candidate subsets.

In some embodiments, at step 410, one or more candidate materials are processed by the recognition composition optimization module 160. In some embodiments, the recognition composition optimization module 160 evaluates elemental selections, stoichiometric combinations, substitutional possibilities, dopant choices, alloy fractions, site occupations, or other composition-related candidate features. In some embodiments, the recognition composition optimization module 160 generates one or more composition-related outputs 168, including one or more composition scores, ranked composition lists, filtered composition subsets, or composition-prioritization outputs.

In some embodiments, at step 412, one or more candidate materials are processed by the recognition-based property prediction module 150. In some embodiments, the recognition-based property prediction module 150 uses one or more baseline property values, one or more recognition metrics, and, in some embodiments, one or more weighting factors to produce one or more recognition-weighted property outputs 158. In some embodiments, such property outputs 158 include one or more predicted property values, ranking scores, screening values, prioritization values, thresholding outputs, or combinations thereof.

In some embodiments, at step 414, one or more candidate materials are processed by the recognition-based structure optimization module 140. In some embodiments, the recognition-based structure optimization module 140 receives structural inputs 142 and computes one or more recognition-weighted pairwise values 144 and one or more structure-related outputs 148. In some embodiments, the structure-related outputs 148 include one or more structure scores, structure ranking values, relaxed structures, recognition-weighted structural energies, or combinations thereof. In some embodiments, the ordering of steps 408, 410, 412, and 414 is varied. In some embodiments, stability screening is performed first to reduce the candidate pool. In some embodiments, composition optimization is performed before property prediction. In some embodiments, property prediction is performed before structure optimization. In some embodiments, two or more of the steps are performed in parallel. In some embodiments, one or more of the steps are repeated iteratively as new information becomes available.

In some embodiments, at step 416, outputs from two or more downstream modules are provided to the combined scoring and ranking module 180. In some embodiments, the combined scoring and ranking module 180 computes one or more combined outputs using one or more property-related values, one or more stability-related values, one or more composition-related values, one or more structure-related values, or combinations thereof. In some embodiments, the combined outputs include one or more overall material scores, candidate-priority lists, shortlist outputs, ranked candidate sets, or downstream decision values.

In some embodiments, at step 418, the system 100 selects one or more next actions based on the combined outputs and, in some embodiments, one or more subsystem-specific outputs. In some embodiments, the one or more next actions include selecting candidates for higher-fidelity simulation, selecting candidates for experimental validation, selecting candidates for synthesis, rejecting candidates, storing candidates for future consideration, requesting additional input data, or reprocessing candidates under modified workflow settings.

In some embodiments, at step 420, the system 100 outputs one or more material-analysis outputs 190. In some embodiments, the material-analysis outputs 190 include one or more candidate rankings 192, one or more screening decisions 194, one or more prediction outputs 196, and one or more optimization outputs 198. In some embodiments, the material-analysis outputs 190 are presented through one or more user interface components 350, stored in one or more storage devices 330, transmitted through one or more communication interfaces 340, or provided to one or more external computational platforms 200.

In some embodiments, the workflow 400 is implemented as a single-pass workflow in which each candidate is evaluated once using a selected set of modules. In some embodiments, the workflow 400 is implemented as a staged workflow in which an initial large candidate set is progressively reduced by one or more screening operations. In some embodiments, the workflow 400 is implemented as an iterative workflow in which outputs from one stage are used to update candidate definitions, refresh baseline values, adjust thresholds, or refine downstream ranking criteria.

In some embodiments, the workflow 400 is configured to reduce computational burden by allowing lower-ranked candidates to be filtered before additional computational resources are consumed. In some embodiments, the workflow 400 is configured to provide a unified ranking framework across multiple evaluation domains, including structure, property, composition, and stability. In some embodiments, the workflow 400 provides a common recognition-based decision framework for candidate prioritization even when different candidates originate from different computational engines, data sources, or material classes.

In some embodiments, the workflow 400 is used for high-throughput candidate screening. In some embodiments, the workflow 400 is used for smaller-batch expert-guided analysis. In some embodiments, the workflow 400 is used in a closed-loop discovery environment in which candidate materials selected at step 418 are further simulated, experimentally evaluated, or fabricated, and resulting data are then returned to the system 100 for additional recognition-enhanced processing. In some embodiments, the workflow 400 is applied to a candidate set that includes metals, alloys, semiconductors, ceramics, polymers, composites, battery materials, two-dimensional materials, perovskites, metal-organic frameworks, catalytic materials, coating materials, or combinations thereof. In some embodiments, different categories of candidate materials are processed using different subsets of the modules 140, 150, 160, and 170 while still using the recognition coverage function 122 as a common weighting basis across multiple stages of analysis.

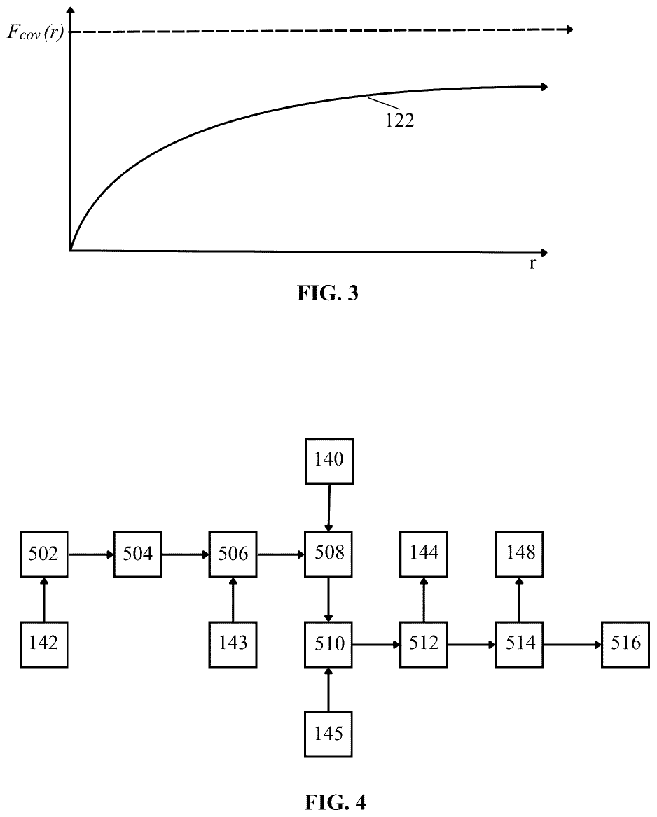

FIG. 3 illustrates the recognition coverage function 122 used in certain embodiments of the present disclosure. In some embodiments, the recognition coverage function 122 provides a bounded and monotone weighting relationship that is applied to one or more nonnegative metrics associated with candidate-material evaluation. In some embodiments, the recognition coverage function 122 is used by one or more of the preprocessing and normalization module 130, the recognition-based structure optimization module 140, the recognition-based property prediction module 150, the recognition composition optimization module 160, the recognition stability screening module 170, and the combined scoring and ranking module 180.

In some embodiments, the recognition coverage function 122 is denoted Fcov(r) and defined as Fcov(r)=r/(r+Xopt), where r is a nonnegative metric and Xopt is a recognition constant. In some embodiments, Xopt=phi/pi, where phi=(1+sqrt(5))/2. In some embodiments, Xopt is treated as a fixed constant that is used across a plurality of recognition-enhanced computational modules so that a common weighting policy is maintained throughout the system 100.

In some embodiments, the nonnegative metric r supplied to the recognition coverage function 122 is a normalized quantity. In some embodiments, r is a normalized distance metric. In some embodiments, r is a normalized interaction metric. In some embodiments, r is a normalized property-related metric. In some embodiments, r is a normalized cohesive-energy-related metric. In some embodiments, r is a normalized stability-related metric. In some embodiments, r is derived from an absolute-value transformation, a magnitude extraction operation, a scaling operation, a clipping operation, a threshold-preparation operation, a reference-based normalization, or combinations thereof.

In some embodiments, the recognition coverage function 122 is zero when the input metric r is zero. In some embodiments, the recognition coverage function 122 increases as the input metric r increases. In some embodiments, the recognition coverage function 122 remains below one for finite nonnegative values of r. In some embodiments, the recognition coverage function 122 approaches one as the input metric r becomes large. In some embodiments, such behavior allows the recognition coverage function 122 to provide continuous weighting without unbounded amplification of the input metric.

In some embodiments, the recognition coverage function 122 is used as a weighting factor rather than as a replacement for the underlying baseline metric itself. In some embodiments, the baseline metric retains its original role as an input derived from simulation, modeling, or measurement, while the recognition coverage function 122 provides a bounded modifier that scales or weights the baseline metric according to a corresponding recognition-compatible quantity. In some embodiments, this permits the disclosed framework to operate as a post-processing or augmentation layer on top of existing computational outputs.

In some embodiments, the recognition coverage function 122 is applied to dimensionless quantities. In some embodiments, the recognition coverage function 122 is applied to quantities that are rendered dimensionless through normalization relative to one or more reference values. In some embodiments, the one or more reference values include a reference distance, a reference energy, a reference property value, a reference cohesive-energy magnitude, a reference stability value, or another selected normalization quantity. In some embodiments, use of normalized inputs provides a consistent basis for applying the recognition coverage function 122 across multiple material classes and multiple computational tasks.

In some embodiments, different categories of raw inputs are converted into nonnegative recognition-compatible metrics before application of the recognition coverage function 122. For example, in some embodiments, a signed energy value is converted into a magnitude-based metric using an absolute-value operation. In some embodiments, a signed value is transformed into a shifted positive quantity. In some embodiments, a ratio is formed relative to a reference magnitude. In some embodiments, a logarithmic or other monotone transform is applied before normalization. In some embodiments, the selected transform depends on the type of material quantity being processed and on whether the downstream module operates on magnitude, sign-preserved value, or another representation.

In some embodiments, the recognition coverage function 122 is used in a parameter-light manner. In some embodiments, the recognition coverage function 122 is used with Xopt as a fixed constant and without introducing additional recognition-specific fitting parameters. In some embodiments, other task-level coefficients, thresholds, or weighting values are still used within the system 100, but the core recognition coverage relationship remains the same across multiple modules. In some embodiments, this provides a common mathematical basis for structure-related processing, property-related processing, composition-related processing, and stability-related processing. In some embodiments, the recognition coverage function 122 is used to support candidate comparison across different analysis stages. In some embodiments, a first candidate material and a second candidate material may be evaluated using different baseline quantities but the resulting recognition coverage values may still be used within a common bounded scoring framework. In some embodiments, this permits downstream modules to contribute to a combined ranking process without requiring that all underlying raw quantities be of the same physical type or unit system.

In some embodiments, the graph shown in FIG. 3 illustrates that relatively small values of the input metric r produce relatively small recognition coverage values, while larger values of the input metric r produce progressively larger recognition coverage values with diminishing incremental increase as the function approaches an upper bound. In some embodiments, this bounded response is useful in avoiding runaway score amplification while still preserving ordered differentiation among candidates having different underlying metrics.

In some embodiments, the recognition coverage function 122 is evaluated repeatedly across a set of candidate materials or across a set of metrics associated with a single candidate material. In some embodiments, the resulting recognition coverage values are stored as part of the recognition metrics 336 in the storage devices 330. In some embodiments, one or more recognition coverage values are reused across multiple downstream modules. In some embodiments, this reuse reduces redundant computation and supports a unified recognition-enhanced workflow across the system 100. In some embodiments, the recognition coverage function 122 is used in conjunction with one or more weighted sums, aggregated scores, thresholding operations, filtering operations, ranking operations, optimization operations, or combinations thereof. In some embodiments, the recognition coverage function 122 is not limited to a single workflow order or a single module and may instead be applied wherever a bounded recognition-based weighting relationship is useful within the disclosed candidate-material evaluation framework.

In some embodiments, the recognition coverage function 122 provides a common weighting policy that links the modules 140, 150, 160, and 170 into a unified computational framework. In some embodiments, the use of the same recognition coverage function 122 across a plurality of modules improves consistency in how structural metrics, property metrics, composition metrics, and stability metrics are converted into downstream ranking and decision outputs. In some embodiments, this common functional form supports coordinated candidate prioritization across multiple stages of computational materials discovery.

FIG. 4 illustrates an example recognition-based structure optimization process carried out by the recognition-based structure optimization module 140. In some embodiments, the process of FIG. 4 is used to evaluate, rank, relax, compare, or optimize candidate material structures using one or more recognition-weighted structural metrics. In some embodiments, the process of FIG. 4 is applied to bulk crystalline materials, non-crystalline materials, alloys, interfaces, surfaces, defect-containing structures, layered materials, porous materials, molecular solids, polymeric materials, or combinations thereof.

In some embodiments, the process of FIG. 4 begins at step 502, where one or more structural inputs 142 associated with a candidate material are received by the recognition-based structure optimization module 140. In some embodiments, the structural inputs 142 include atomic coordinates, lattice vectors, unit-cell parameters, symmetry information, species assignments, pairwise distance values, local-neighborhood definitions, interaction values, bond-related descriptors, graph-representation data, force-related quantities, energy-related quantities, or combinations thereof.

In some embodiments, at step 504, the recognition-based structure optimization module 140 determines one or more distance-related metrics associated with pairs of atoms, sites, nodes, or other structural entities. In some embodiments, a pairwise distance value is determined between a first structural entity and a second structural entity. In some embodiments, the pairwise distance value is represented in direct coordinate space, periodic-image space, graph distance space, local-neighborhood space, or another structure-associated distance representation. In some embodiments, periodic boundary conditions are taken into account when determining the pairwise distance value. In some embodiments, at step 506, the recognition-based structure optimization module 140 generates one or more normalized distance metrics 143 from the one or more distance-related metrics. In some embodiments, a distance value is normalized relative to a reference distance. In some embodiments, the reference distance is a nearest-neighbor distance, a lattice-derived distance, an empirically selected reference, a class-specific structural reference, or another selected normalization value. In some embodiments, the normalized distance metric 143 is nonnegative and is suitable for direct processing by the recognition coverage function 122.

In some embodiments, at step 508, the recognition-based structure optimization module 140 evaluates the recognition coverage function 122 using the normalized distance metric 143. In some embodiments, the recognition coverage value corresponds to Fcov(r), where r is the normalized distance metric 143. In some embodiments, the resulting recognition coverage value is stored as part of the recognition-weighted pairwise values 144. In some embodiments, separate recognition coverage values are determined for different neighbor shells, different bond environments, different atom-pair types, or different categories of structural interactions.

In some embodiments, at step 510, the recognition-based structure optimization module 140 receives or determines one or more interaction-related quantities 145 associated with the structural entities under analysis. In some embodiments, the one or more interaction-related quantities 145 include pairwise energy quantities, pairwise cost quantities, pairwise affinity quantities, pairwise force-related quantities, bond-strength-related quantities, graph-edge weights, or other structure-associated interaction metrics. In some embodiments, the one or more interaction-related quantities 145 are obtained from a first-principles engine, a molecular dynamics engine, an interatomic potential, a machine-learning potential, an empirical model, a database value, or another computational or data source.

In some embodiments, at step 512, the recognition-based structure optimization module 140 determines one or more recognition-weighted pairwise values 144 by combining the one or more interaction-related quantities 145 with corresponding recognition coverage values. In some embodiments, a recognition-weighted pairwise value is determined by multiplying an interaction-related quantity by a recognition coverage value. In some embodiments, the recognition-weighted pairwise values 144 represent bounded structural contributions associated with corresponding pairs of atoms, sites, nodes, or structural entities.

In some embodiments, the interaction-related quantity 145 is a nonnegative metric. In some embodiments, the interaction-related quantity 145 represents a magnitude of a pairwise interaction, a cost-like quantity, a bond-strength-like quantity, or another nonnegative structural metric. In some embodiments, the recognition-weighted pairwise value 144 is determined by multiplying the nonnegative interaction-related quantity 145 by the recognition coverage value associated with the corresponding normalized distance metric 143.

In some embodiments, the interaction-related quantity 145 is a signed quantity. In some embodiments, the recognition-based structure optimization module 140 uses a sign-preserving embodiment in which the sign of the interaction-related quantity 145 is preserved while the magnitude of the interaction-related quantity 145 is recognition weighted. In some embodiments, the recognition-based structure optimization module 140 uses a magnitude-only embodiment in which an absolute value or other nonnegative transform of the interaction-related quantity 145 is used to generate the recognition-weighted pairwise value 144. In some embodiments, the selected embodiment depends on whether the downstream structure-related output is intended to represent a magnitude-based score, a signed structure-energy-related quantity, or another structure-related value. In some embodiments, at step 514, the recognition-based structure optimization module 140 aggregates the recognition-weighted pairwise values 144 to generate one or more structure-related outputs 148. In some embodiments, the recognition-weighted pairwise values 144 are summed across a candidate structure. In some embodiments, the recognition-weighted pairwise values 144 are summed across selected neighbor shells. In some embodiments, the recognition-weighted pairwise values 144 are combined using one or more weighted sums, averages, class-specific aggregation rules, graph-based aggregation operations, or combinations thereof.

In some embodiments, the structure-related outputs 148 include a structure score and one or more additional structure-related outputs, such as a structure ranking value, a recognition-weighted structure energy, a recognition-weighted pairwise map, a local-environment score, a relaxed structure output, or combinations thereof. In some embodiments, the structure score is used to compare candidate structures. In some embodiments, the recognition-weighted structure energy is used as an objective or pseudo-objective in a downstream optimization procedure. In some embodiments, the local-environment score is used to identify regions of a structure that contribute disproportionately to a structural ranking or instability.

In some embodiments, at step 516, one or more optimization operations are performed using the structure-related outputs 148. In some embodiments, the optimization operations include coordinate relaxation, lattice relaxation, defect-position adjustment, site-occupation modification, interface reconfiguration, layer-stacking adjustment, graph-structure refinement, or other structure-update operations. In some embodiments, the optimization operation is carried out using gradient descent, conjugate-gradient optimization, quasi-Newton optimization, simulated annealing, genetic search, Bayesian search, or another optimization technique.

In some embodiments, when a coordinate- or geometry-updating optimization is used, the recognition-based structure optimization module 140 recalculates one or more distance-related metrics, one or more normalized distance metrics 143, and one or more recognition-weighted pairwise values 144 after a structural update. In some embodiments, the process of FIG. 4 is therefore iterative and repeats steps 504 through 516 until a stopping condition is met. In some embodiments, the stopping condition includes convergence of a structure-related output 148, convergence of an objective value, convergence of an update magnitude, a maximum iteration count, a threshold change criterion, or another termination condition.

In some embodiments, the process of FIG. 4 is used as a post-processing evaluation procedure rather than as a direct geometry-update procedure. In some embodiments, the structure-related outputs 148 are used only for ranking, screening, filtering, or prioritizing candidate structures after a separate external engine has already performed structure relaxation. In some embodiments, the process of FIG. 4 is therefore compatible with workflows in which an external first-principles engine or molecular dynamics engine supplies structural inputs 142 and the recognition-based structure optimization module 140 applies recognition-enhanced scoring without altering the source code of the external engine.

In some embodiments, the process of FIG. 4 is applied using full pairwise enumeration. In some embodiments, the process of FIG. 4 is applied using only nearest-neighbor interactions. In some embodiments, the process of FIG. 4 is applied using a shell-based truncation policy, a cutoff-distance policy, a graph-neighborhood policy, or a sparse-pair evaluation policy. In some embodiments, the selection of which pairs are processed depends on computational budget, material type, structure size, or target fidelity.

In some embodiments, the recognition-based structure optimization module 140 is used to compare multiple structural candidates corresponding to the same composition. In some embodiments, the recognition-based structure optimization module 140 is used to compare defect configurations, phase variants, interface arrangements, surface terminations, dopant placements, or polymorph candidates. In some embodiments, the recognition-based structure optimization module 140 is used to determine which candidate structures should be advanced to one or more downstream modules or to higher-fidelity simulation.

In some embodiments, the structure-related outputs 148 generated by the process of FIG. 4 are provided to the combined scoring and ranking module 180. In some embodiments, one or more structure-related outputs 148 are combined with one or more property-related outputs 158, one or more composition-related outputs 168, and one or more stability-related outputs 178 to determine an overall candidate-material ranking. In some embodiments, the structure-related outputs 148 are also stored in the storage devices 330 as part of the candidate-material records 332 or ranking results 338. In some embodiments, the process of FIG. 4 provides a bounded structure-evaluation framework in which distance-related information and interaction-related information are integrated using the recognition coverage function 122. In some embodiments, the process of FIG. 4 provides a consistent structure-related weighting policy that can be used across different material classes and different computational workflows while remaining compatible with external sources of structural and interaction-related input data.

FIG. 5 illustrates an example pairwise interaction evaluation arrangement used in certain embodiments of the recognition-based structure optimization module 140. In some embodiments, the pairwise interaction evaluation arrangement provides a more detailed view of how structural entities within a candidate material are processed to generate one or more recognition-weighted pairwise values 144 and one or more resulting structure-related outputs 148.

In the embodiment shown in FIG. 5, a candidate structure includes a plurality of structural entities. In some embodiments, the structural entities are atoms. In some embodiments, the structural entities are lattice sites, graph nodes, bonded units, molecular fragments, coarse-grained regions, or other structure-associated entities. In some embodiments, a selected structural entity 606 is evaluated relative to one or more neighboring structural entities 608, 610, 612, and 614. In some embodiments, the neighboring structural entities 608, 610, 612, and 614 correspond to different bond environments, different neighbor shells, different chemical species, different coordination environments, or different periodic images.

In some embodiments, one or more pairwise separation values 620 are determined between the selected structural entity 606 and the neighboring structural entities 608, 610, 612, and 614. In some embodiments, the one or more pairwise separation values 620 include respective separation values associated with the neighboring structural entities 608, 610, 612, and 614. In some embodiments, a first separation value corresponds to a separation between the selected structural entity 606 and the neighboring structural entity 608, a second separation value corresponds to a separation between the selected structural entity 606 and the neighboring structural entity 610, a third separation value corresponds to a separation between the selected structural entity 606 and the neighboring structural entity 612, and a fourth separation value corresponds to a separation between the selected structural entity 606 and the neighboring structural entity 614.

In some embodiments, the one or more pairwise separation values 620 are provided to a distance-normalization stage 630. In some embodiments, the distance-normalization stage 630 generates one or more normalized distance metrics 632 using one or more reference distances 634. In some embodiments, the one or more reference distances 634 include a nearest-neighbor reference distance, a lattice-derived reference distance, an average bond-length reference, a species-pair reference distance, or another selected normalization quantity. In some embodiments, the normalized distance metrics 632 include respective normalized distance metrics corresponding to the pairwise separation values 620.

In some embodiments, the normalized distance metrics 632 are supplied to a recognition coverage evaluation stage 650. In some embodiments, the recognition coverage evaluation stage 650 applies the recognition coverage function 122 to the normalized distance metrics 632 to generate one or more recognition coverage values 652. In some embodiments, the recognition coverage values 652 include respective recognition coverage values corresponding to the normalized distance metrics 632.

In some embodiments, one or more interaction-related quantities 670 are also determined for the pairwise relationships associated with the selected structural entity 606. In some embodiments, the interaction-related quantities 670 include respective interaction quantities associated with the pairwise relationships under evaluation. In some embodiments, the interaction-related quantities 670 are pairwise energy values, bond-strength values, force-related values, cost-like values, graph-edge weights, affinity values, or other structure-associated pairwise metrics. In some embodiments, the interaction-related quantities 670 are obtained from one or more external computational platforms 200. In some embodiments, the interaction-related quantities 670 are determined internally by the system 100.

In some embodiments, the recognition coverage values 652 and the interaction-related quantities 670 are supplied to a pairwise weighting stage 680. In some embodiments, the pairwise weighting stage 680 determines one or more recognition-weighted pairwise values 682. In some embodiments, the recognition-weighted pairwise values 682 include respective recognition-weighted pairwise values generated by combining corresponding interaction-related quantities with corresponding recognition coverage values.

In some embodiments, the pairwise weighting stage 680 uses a nonnegative-magnitude embodiment. In some embodiments, a nonnegative interaction magnitude is multiplied by a corresponding recognition coverage value to determine a corresponding recognition-weighted pairwise value. In some embodiments, the nonnegative-magnitude embodiment is used when a resulting structure-related output is intended to represent a magnitude-based score, a bounded structural contribution, or another nonnegative structure-related quantity.

In some embodiments, the pairwise weighting stage 680 uses a sign-preserving embodiment. In some embodiments, the sign of a signed interaction-related quantity is preserved while the magnitude of the signed interaction-related quantity is recognition weighted. In some embodiments, the sign-preserving embodiment is used when the resulting structure-related output is intended to represent a signed structure-energy-related quantity or another signed structural value. In some embodiments, the pairwise weighting stage 680 selectively applies the nonnegative-magnitude embodiment to some pairwise quantities and the sign-preserving embodiment to other pairwise quantities within the same candidate evaluation workflow.

In some embodiments, the recognition-weighted pairwise values 682 are supplied to an aggregation stage 700. In some embodiments, the aggregation stage 700 determines one or more local structural outputs 702 and one or more global structural outputs 704. In some embodiments, the one or more local structural outputs 702 include a local environment score, a local coordination contribution, a local structural priority value, or combinations thereof. In some embodiments, the one or more global structural outputs 704 include a structure score, a recognition-weighted structure energy, a structure ranking value, or combinations thereof.

In some embodiments, the aggregation stage 700 aggregates recognition-weighted pairwise values associated with a single selected structural entity 606 to generate the one or more local structural outputs 702. In some embodiments, the aggregation stage 700 aggregates recognition-weighted pairwise values associated with a plurality of structural entities to generate the one or more global structural outputs 704. In some embodiments, separate aggregations are performed for different atom types, different coordination shells, different graph neighborhoods, different periodic images, different crystal directions, or different defect-associated regions.

In some embodiments, the local structural outputs 702 are used to identify one or more local regions of interest 720 within the candidate structure. In some embodiments, the one or more local regions of interest 720 correspond to defect neighborhoods, strained regions, under-coordinated sites, substitution sites, interface zones, surface regions, adsorption sites, or other local environments that materially affect structural ranking or optimization. In some embodiments, the local structural outputs 702 are used to guide a downstream structure update, dopant-placement decision, defect-selection step, or interface-modification step.

In some embodiments, the global structural outputs 704 are supplied to an optimization or ranking stage 730. In some embodiments, the optimization or ranking stage 730 uses the global structural outputs 704 to compare multiple candidate structures, prioritize one or more candidate structures for downstream analysis, reject one or more lower-ranked candidates, or initiate one or more structural refinement operations. In some embodiments, the optimization or ranking stage 730 outputs one or more ranked structure candidates, one or more refinement instructions, or one or more downstream handoff outputs.

In some embodiments, the pairwise interaction evaluation arrangement is applied to periodic crystalline materials. In some embodiments, the pairwise interaction evaluation arrangement is applied to amorphous materials. In some embodiments, the pairwise interaction evaluation arrangement is applied to molecular crystals, layered materials, interfaces, surfaces, defect-containing materials, or other structures in which local pairwise relationships provide useful structural discrimination.

In some embodiments, the pairwise interaction evaluation arrangement is implemented using direct pairwise enumeration. In some embodiments, the pairwise interaction evaluation arrangement is implemented using neighbor lists, graph adjacency lists, shell-based groupings, sparse edge sets, or cutoff-based pair selection. In some embodiments, such implementation choices are based on structure size, computational budget, desired fidelity, or availability of upstream structural information.

In some embodiments, the pairwise interaction evaluation arrangement provides a more granular structural-analysis layer than the broader workflow of FIG. 4. In some embodiments, FIG. 4 emphasizes the process-level flow performed by the recognition-based structure optimization module 140, whereas FIG. 5 emphasizes how pairwise structural relationships are transformed into local and global structure-related outputs through normalization, recognition weighting, and aggregation.

In some embodiments, one or more outputs produced by the pairwise interaction evaluation arrangement are stored as recognition metrics 336, candidate-material records 332, or ranking results 338. In some embodiments, one or more outputs produced by the pairwise interaction evaluation arrangement are supplied to the combined scoring and ranking module 180, to the recognition-based property prediction module 150, or to another downstream module of the system 100. In some embodiments, this allows pairwise structural analysis to contribute directly to broader candidate-material prioritization decisions.

In some embodiments, the pairwise interaction evaluation arrangement allows the system 100 to distinguish among structurally similar candidate materials by applying a common bounded recognition-weighting policy to multiple pairwise relationships within each candidate structure. In some embodiments, the resulting local and global structural outputs support more informed ranking, screening, and optimization decisions across candidate materials having different geometries, environments, or structural complexities.

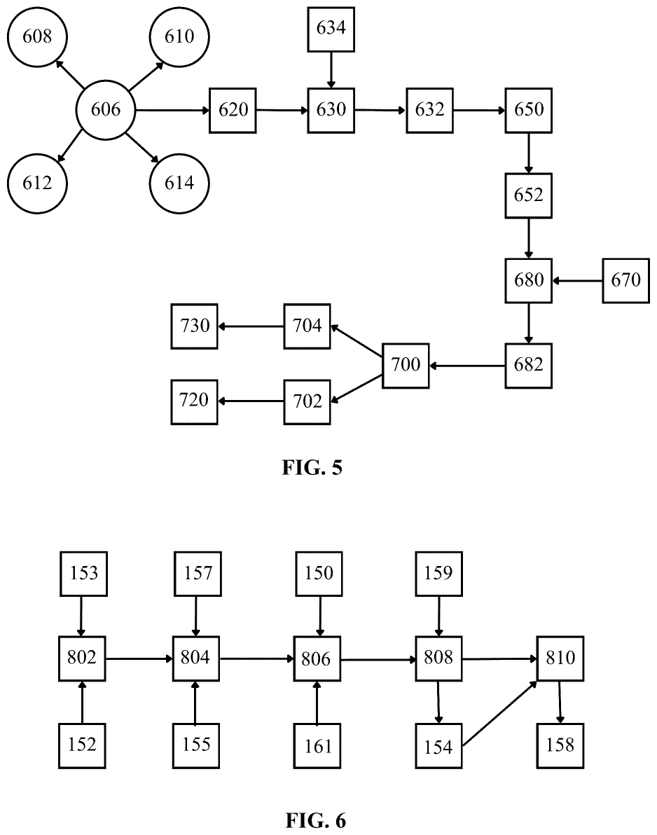

FIG. 6 illustrates an example recognition-based property prediction process carried out by the recognition-based property prediction module 150. In some embodiments, the process of FIG. 6 is used to generate one or more recognition-weighted property outputs 158 for a candidate material using one or more baseline property values, one or more recognition metrics, and, in some embodiments, one or more weighting factors. In some embodiments, the process of FIG. 6 is used for prediction, ranking, screening, thresholding, prioritization, or combinations thereof.

In some embodiments, the process of FIG. 6 begins at step 802, where one or more property inputs 152 associated with a candidate material are received. In some embodiments, the one or more property inputs 152 include one or more baseline property values 153, one or more property descriptors 155, one or more recognition metrics 157, and, in some embodiments, one or more weighting factors 159. In some embodiments, the one or more baseline property values 153 are obtained from one or more first-principles engines, one or more molecular dynamics engines, one or more machine-learning models, one or more empirical models, one or more databases, one or more experimentally derived datasets, or combinations thereof.

In some embodiments, the one or more baseline property values 153 correspond to electronic properties, optical properties, mechanical properties, thermal properties, magnetic properties, transport properties, electrochemical properties, chemical properties, catalytic properties, structural descriptors, or combinations thereof. In some embodiments, the one or more baseline property values 153 include one or more bandgap values, elastic modulus values, hardness values, conductivity values, diffusivity values, dielectric values, refractive-index values, adsorption-energy-related values, capacity values, redox-related values, charge-transport values, thermal-conductivity values, Seebeck values, or combinations thereof.

In some embodiments, at step 804, the recognition-based property prediction module 150 determines one or more recognition metrics 157 associated with the one or more baseline property values 153. In some embodiments, a recognition metric 157 is based on a normalized property quantity. In some embodiments, a recognition metric 157 is based on a ratio between a property-related value and a reference value. In some embodiments, a recognition metric 157 is based on a normalized cohesive-energy-related value, a structural descriptor, a density-related quantity, a defect-related quantity, a composition-related descriptor, a stability-related quantity, or another quantity associated with property interpretation or ranking.

In some embodiments, the recognition metrics 157 are generated by the preprocessing and normalization module 130 before being supplied to the recognition-based property prediction module 150. In some embodiments, the recognition-based property prediction module 150 generates the recognition metrics 157 internally. In some embodiments, each recognition metric 157 is nonnegative and is suitable for direct processing by the recognition coverage function 122. In some embodiments, a signed raw property-related quantity is converted into a nonnegative metric using an absolute-value transform, a shifted-positive transform, a normalized magnitude transform, or another transform suitable for producing a recognition-compatible input.

In some embodiments, at step 806, the recognition-based property prediction module 150 applies the recognition coverage function 122 to the one or more recognition metrics 157 to determine one or more recognition coverage values 161. In some embodiments, each recognition coverage value 161 corresponds to a respective recognition metric 157. In some embodiments, the one or more recognition coverage values 161 are used as bounded weighting values associated with corresponding property-related terms.

In some embodiments, at step 808, the recognition-based property prediction module 150 determines one or more recognition-weighted property terms 154. In some embodiments, a recognition-weighted property term 154 is determined by combining a baseline property value 153, a recognition coverage value 161, and, in some embodiments, a weighting factor 159. In some embodiments, a recognition-weighted property term 154 is determined by multiplying a property weighting factor, a baseline property value, and a recognition coverage value derived from a corresponding recognition metric. In some embodiments, the one or more recognition-weighted property terms 154 include a plurality of property terms associated with a selected candidate-material evaluation task. In some embodiments, the weighting factor 159 is a fixed value. In some embodiments, the weighting factor 159 is selected by a user. In some embodiments, the weighting factor 159 is selected based on material class, target application, property family, workflow stage, confidence level, or optimization objective. In some embodiments, the weighting factor 159 is omitted, and the recognition-weighted property term 154 is determined using only the baseline property value 153 and the corresponding recognition coverage value 161.

In some embodiments, at step 810, the recognition-based property prediction module 150 aggregates the one or more recognition-weighted property terms 154 to generate one or more property-related outputs 158. In some embodiments, the one or more property-related outputs 158 include a predicted property value, a property ranking score, a screening value, a thresholding value, a prioritization value, or combinations thereof. In some embodiments, the predicted property value is a recognition-weighted property estimate. In some embodiments, the property ranking score is used to compare multiple candidate materials. In some embodiments, the screening value is used to determine whether a candidate is passed to a downstream workflow stage.

In some embodiments, the one or more recognition-weighted property terms 154 are summed to determine the predicted property value. In some embodiments, the one or more recognition-weighted property terms 154 are combined using a weighted average, a normalized weighted sum, a threshold-conditioned aggregation, a class-specific aggregation rule, or another aggregation policy. In some embodiments, the selected aggregation policy depends on the property type, the material class, the number of property channels being considered, or the workflow objective.

In some embodiments, the process of FIG. 6 is used for single-property screening. In some embodiments, the process of FIG. 6 is used for multi-property screening. In some embodiments, the process of FIG. 6 is used to jointly evaluate two or more candidate-material characteristics, such as bandgap and stability, conductivity and diffusivity, hardness and oxidation resistance, or capacity and electrochemical stability. In some embodiments, separate property-related outputs 158 are generated for different property families and are later supplied to the combined scoring and ranking module 180.

In some embodiments, the process of FIG. 6 is used primarily for ranking rather than for direct substitution of the baseline property value 153. In some embodiments, the recognition-based property prediction module 150 generates a recognition-weighted score that assists in candidate prioritization while the baseline property value 153 remains available as a separate reported quantity. In some embodiments, this allows the system 100 to preserve provenance and interpretability while still applying recognition-enhanced ranking logic across a candidate set.

In some embodiments, the process of FIG. 6 uses a confidence-aware embodiment. In some embodiments, the one or more property inputs 152 include one or more confidence values, uncertainty values, variance values, model-disagreement values, or data-quality indicators. In some embodiments, the recognition-based property prediction module 150 uses such information to adjust the weighting factor 159, adjust one or more threshold values, flag one or more outputs for review, or generate one or more confidence-qualified property-related outputs 158.

In some embodiments, the process of FIG. 6 uses one or more reference values for normalization. In some embodiments, the one or more reference values include a family-specific baseline property, a literature benchmark, a database-derived comparison value, a user-selected target value, or another normalization quantity. In some embodiments, the one or more reference values are selected so that candidate materials from different classes or different source datasets can be evaluated within a consistent recognition-weighted framework.

In some embodiments, the process of FIG. 6 is applied to semiconductor candidate materials to generate one or more bandgap-related ranking outputs. In some embodiments, the process of FIG. 6 is applied to battery candidate materials to generate one or more conductivity-related or capacity-related outputs. In some embodiments, the process of FIG. 6 is applied to alloy candidate materials to generate one or more hardness-related or modulus-related outputs. In some embodiments, the process of FIG. 6 is applied to catalytic materials, coating materials, optical materials, magnetic materials, polymer materials, composite materials, or other candidate-material classes.

In some embodiments, the one or more property-related outputs 158 generated by the process of FIG. 6 are provided to the combined scoring and ranking module 180. In some embodiments, the one or more property-related outputs 158 are also stored in the candidate-material records 332, the recognition metrics 336, or the ranking results 338. In some embodiments, the one or more property-related outputs 158 are provided to a user through the user interface components 350, exported through the communication interfaces 340, or used to trigger one or more downstream computational or experimental actions.

In some embodiments, the process of FIG. 6 is carried out after one or more lower-priority candidate materials have already been removed by the recognition stability screening module 170 or the recognition composition optimization module 160. In some embodiments, the process of FIG. 6 is carried out before structure optimization so that only property-promising candidates are passed to the recognition-based structure optimization module 140. In some embodiments, the process of FIG. 6 is iteratively repeated as new baseline property values become available from updated simulations, revised models, or newly acquired experimental data.

In some embodiments, the process of FIG. 6 provides a bounded recognition-enhanced property-evaluation framework in which baseline property values are combined with recognition-compatible metrics to generate outputs useful for ranking and prioritization. In some embodiments, the process of FIG. 6 improves comparability across candidates by applying a common recognition coverage function 122 to one or more property-associated metrics while retaining flexibility in the selection of baseline property values, normalization policies, and aggregation rules.

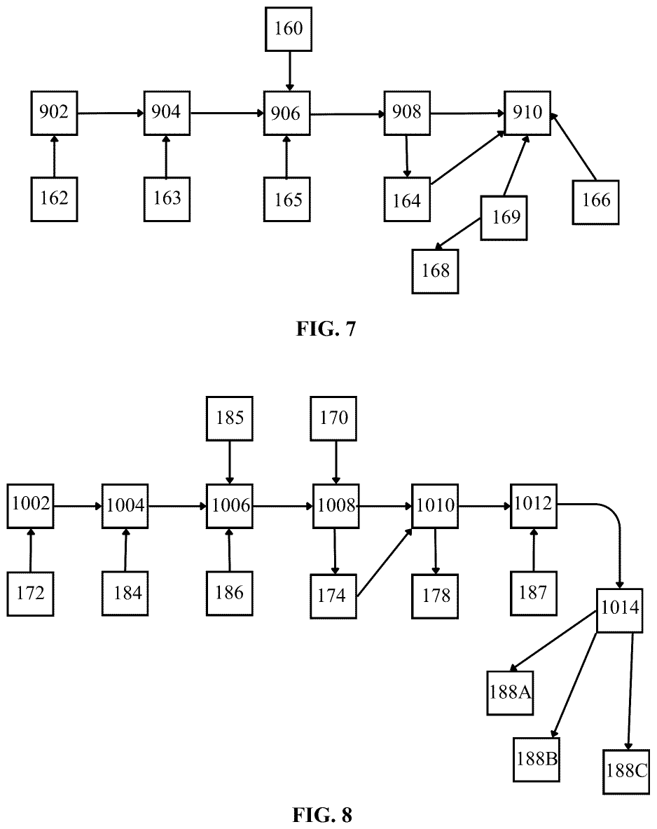

FIG. 7 illustrates an example recognition composition optimization process carried out by the recognition composition optimization module 160. In some embodiments, the process of FIG. 7 is used to evaluate, rank, filter, or optimize candidate material compositions using one or more recognition-weighted composition values. In some embodiments, the process of FIG. 7 is applied to binary systems, ternary systems, quaternary systems, multicomponent alloys, doped materials, substituted crystal families, layered materials, battery materials, semiconductor materials, catalytic materials, coating materials, polymer systems, composite systems, or other candidate material classes in which composition selection affects downstream material performance.

In some embodiments, the process of FIG. 7 begins at step 902, where one or more composition inputs 162 associated with a candidate material are received by the recognition composition optimization module 160. In some embodiments, the one or more composition inputs 162 include elemental identities, stoichiometric fractions, site assignments, host-species definitions, dopant identities, dopant fractions, substitution options, vacancy options, alloy fractions, precursor definitions, or combinations thereof. In some embodiments, the one or more composition inputs 162 are obtained from a user, generated by a candidate-enumeration routine, imported from a database, produced by an upstream design tool, or obtained from another computational workflow.

In some embodiments, at step 904, the recognition composition optimization module 160 determines one or more composition-associated metrics 163 for the one or more composition inputs 162. In some embodiments, the one or more composition-associated metrics 163 include cohesive-energy-related quantities, binding-related quantities, compatibility-related quantities, size-mismatch quantities, valence-related quantities, electronegativity-related quantities, site-preference-related quantities, solubility-related quantities, phase-compatibility-related quantities, or combinations thereof. In some embodiments, the one or more composition-associated metrics 163 are determined for individual elements, for pairs of elements, for site-specific occupants, or for larger composition subsets.

In some embodiments, the one or more composition-associated metrics 163 are received from one or more external computational platforms 200. In some embodiments, the one or more composition-associated metrics 163 are obtained from one or more databases or curated repositories. In some embodiments, the one or more composition-associated metrics 163 are determined internally by the system 100 using one or more preprocessing, lookup, normalization, or estimation operations. In some embodiments, the one or more composition-associated metrics 163 are converted into nonnegative recognition-compatible quantities before downstream recognition processing.

In some embodiments, at step 906, the recognition composition optimization module 160 generates one or more normalized composition metrics 165 from the one or more composition-associated metrics 163. In some embodiments, normalization is performed relative to one or more reference quantities, such as a reference cohesive-energy magnitude, a reference compatibility value, a reference stoichiometric quantity, a class-specific baseline, or another selected normalization value. In some embodiments, the one or more normalized composition metrics 165 are nonnegative and are suitable for evaluation by the recognition coverage function 122.

In some embodiments, at step 908, the recognition composition optimization module 160 applies the recognition coverage function 122 to the one or more normalized composition metrics 165 to determine one or more recognition-weighted composition values 164. In some embodiments, a recognition-weighted composition value 164 is associated with a respective element, dopant, site occupant, composition fraction, or other composition constituent. In some embodiments, the recognition-weighted composition values 164 include a plurality of recognition-weighted composition values as needed for a selected candidate composition.

In some embodiments, at step 910, the recognition composition optimization module 160 combines the recognition-weighted composition values 164 with one or more composition fractions 166 to determine one or more composition-related outputs 168. In some embodiments, the one or more composition fractions 166 correspond to stoichiometric fractions, site-occupancy fractions, alloy fractions, dopant concentrations, substitution fractions, or other constituent proportions. In some embodiments, a composition-related output 168 is determined by combining a recognition-weighted composition value associated with a constituent and a corresponding composition fraction associated with that constituent.

In some embodiments, the one or more composition-related outputs 168 include a composition score, a composition ranking value, a composition filter output, a preferred-composition subset, a site-assignment recommendation, or combinations thereof. In some embodiments, the composition score is used to compare multiple candidate compositions. In some embodiments, the composition ranking value is used to order candidates for downstream screening or simulation. In some embodiments, the composition filter output is used to eliminate lower-priority candidates before more computationally intensive analysis is performed.

In some embodiments, the process of FIG. 7 is carried out using an element-by-element embodiment. In some embodiments, each element or constituent in a candidate composition is assigned a recognition-weighted composition value 164, and the recognition-weighted composition values 164 are then combined according to the corresponding composition fractions 166. In some embodiments, the process of FIG. 7 is carried out using a site-specific embodiment in which different crystallographic sites, layer positions, interface regions, or local chemical environments are treated separately when determining the one or more recognition-weighted composition values 164. In some embodiments, the process of FIG. 7 is carried out using a dopant-selection embodiment. In some embodiments, a host material is fixed and a plurality of dopant candidates are evaluated according to one or more recognition-weighted composition values 164. In some embodiments, the process of FIG. 7 is used to rank dopant identities, dopant concentrations, or dopant site preferences before downstream property simulation or experimental validation. In some embodiments, the process of FIG. 7 is carried out using a substitution embodiment in which one or more substitution candidates are evaluated for one or more sites within a candidate structure.

In some embodiments, the process of FIG. 7 is applied to multicomponent alloys. In some embodiments, the process of FIG. 7 is used to evaluate high-entropy alloy candidates, medium-entropy alloy candidates, refractory alloy candidates, corrosion-resistant alloy candidates, or other multicomponent metallic systems. In some embodiments, the process of FIG. 7 is used to compare elemental combinations and fractional compositions prior to downstream structural analysis, thermodynamic analysis, or mechanical-property evaluation.

In some embodiments, the process of FIG. 7 is applied to battery materials. In some embodiments, the process of FIG. 7 is used to evaluate host compositions, dopant compositions, cathode compositions, anode compositions, electrolyte compositions, or coating compositions. In some embodiments, the process of FIG. 7 is applied to semiconductor materials, catalytic materials, optical materials, ceramic materials, polymer materials, composite materials, coating materials, and layered materials. In some embodiments, the process of FIG. 7 is used to reduce the size of a combinatorially large candidate composition space before downstream simulation.

In some embodiments, one or more constraints 169 are applied during or after the determination of the composition-related outputs 168. In some embodiments, the one or more constraints 169 include charge-neutrality constraints, valence-balance constraints, site-capacity constraints, process-compatibility constraints, cost constraints, abundance constraints, manufacturability constraints, toxicity constraints, or combinations thereof. In some embodiments, a candidate composition having a favorable composition score may still be excluded if one or more applicable constraints 169 are not satisfied.

In some embodiments, the composition-related outputs 168 generated by the process of FIG. 7 are provided to the combined scoring and ranking module 180. In some embodiments, the composition-related outputs 168 are also provided to the recognition-based property prediction module 150, the recognition stability screening module 170, or the recognition-based structure optimization module 140. In some embodiments, this allows composition-related screening to be used as an upstream reduction stage before structural, stability, or property analysis is performed.

In some embodiments, the composition-related outputs 168 are stored in the candidate-material records 332, the recognition metrics 336, or the ranking results 338. In some embodiments, the composition-related outputs 168 are displayed through the user interface components 350 or transmitted through the communication interfaces 340. In some embodiments, the composition-related outputs 168 are used to generate one or more reduced candidate sets for downstream simulation, fabrication, or experimental validation.

In some embodiments, the process of FIG. 7 is iterative. In some embodiments, one or more candidate compositions are refined after downstream structural, property, or stability results become available. In some embodiments, a first candidate subset is generated using the recognition composition optimization module 160, after which one or more candidates from the first candidate subset are modified, expanded, or recombined to produce a second candidate subset for additional recognition-enhanced evaluation.

In some embodiments, the process of FIG. 7 provides a bounded and scalable composition-evaluation framework in which one or more composition-associated metrics are processed through the recognition coverage function 122 to generate composition-related outputs useful for ranking and prioritization. In some embodiments, the process of FIG. 7 provides a common recognition-based composition layer that can be used across different material classes and across different levels of composition complexity while remaining compatible with external databases, predictive tools, and simulation workflows.

FIG. 8 illustrates an example recognition stability screening process carried out by the recognition stability screening module 170. In some embodiments, the process of FIG. 8 is used to evaluate, rank, filter, or prioritize candidate materials according to one or more stability-related metrics. In some embodiments, the process of FIG. 8 is used as an early-stage screening operation so that lower-priority candidates are removed or deprioritized before more computationally intensive downstream analysis is performed.

In some embodiments, the process of FIG. 8 begins at step 1002, where one or more stability inputs 172 associated with a candidate material are received by the recognition stability screening module 170. In some embodiments, the one or more stability inputs 172 include one or more formation-enthalpy-related values, one or more energy-above-hull values, one or more decomposition-energy values, one or more free-energy values, one or more phase-stability values, one or more metastability values, or combinations thereof. In some embodiments, the one or more stability inputs 172 are obtained from one or more external computational platforms 200, one or more databases, one or more experimentally derived datasets, or combinations thereof.

In some embodiments, at step 1004, the recognition stability screening module 170 determines one or more stability-associated metrics 184 from the one or more stability inputs 172. In some embodiments, the one or more stability-associated metrics 184 are based on absolute-value transforms, magnitude-based transforms, normalized transforms, reference-based transforms, or combinations thereof. In some embodiments, the one or more stability-associated metrics 184 are nonnegative metrics suitable for evaluation by the recognition coverage function 122.

In some embodiments, a first stability-associated metric 184 is derived from a formation enthalpy quantity. In some embodiments, a second stability-associated metric 184 is derived from an energy-above-hull quantity. In some embodiments, a third stability-associated metric 184 is derived from a decomposition-energy quantity. In some embodiments, a fourth stability-associated metric 184 is derived from a free-energy quantity. In some embodiments, the recognition stability screening module 170 selects one or more of such stability-associated metrics 184 depending on the material class, the available data, the workflow stage, or the desired screening policy.

In some embodiments, at step 1006, the recognition stability screening module 170 generates one or more normalized stability metrics 185 from the one or more stability-associated metrics 184. In some embodiments, normalization is performed relative to one or more reference values 186. In some embodiments, the one or more reference values 186 include a reference formation enthalpy magnitude, a reference stability threshold, a class-specific baseline, a database-derived reference value, or another selected normalization quantity. In some embodiments, the one or more normalized stability metrics 185 are nonnegative and are suitable for direct evaluation by the recognition coverage function 122.

In some embodiments, at step 1008, the recognition stability screening module 170 applies the recognition coverage function 122 to the one or more normalized stability metrics 185 to determine one or more recognition-weighted stability values 174. In some embodiments, each recognition-weighted stability value 174 corresponds to a respective stability-associated metric 184 or normalized stability metric 185. In some embodiments, the one or more recognition-weighted stability values 174 include a plurality of recognition-weighted stability values as needed for a selected workflow.

In some embodiments, at step 1010, the recognition stability screening module 170 determines one or more stability-related outputs 178 from the one or more recognition-weighted stability values 174. In some embodiments, the one or more stability-related outputs 178 include a stability score, a thresholding output, a filtered candidate subset, a pass-rate output, a stability ranking output, or combinations thereof. In some embodiments, the stability score is used to compare multiple candidate materials. In some embodiments, the thresholding output indicates whether a candidate is passed, flagged, deprioritized, or rejected for a downstream stage.

In some embodiments, the stability score is determined by applying the recognition coverage function 122 to a nonnegative stability-associated metric 184. In some embodiments, the stability score is determined using a formation-enthalpy-based metric in which an absolute value of a formation enthalpy quantity is used. In some embodiments, the stability score is determined using an energy-above-hull-based metric. In some embodiments, the stability score is determined using a decomposition-energy-based metric. In some embodiments, the stability score is determined using a combination of two or more stability-associated metrics 184.

In some embodiments, the process of FIG. 8 uses a single-metric embodiment. In some embodiments, a candidate material is assigned one stability score based on one selected stability-associated metric 184. In some embodiments, the process of FIG. 8 uses a multi-metric embodiment in which two or more recognition-weighted stability values 174 are combined to determine the stability score or another stability-related output 178. In some embodiments, the multi-metric embodiment is used when different stability inputs are available for different subsets of candidate materials.

In some embodiments, at step 1012, the recognition stability screening module 170 compares the one or more stability-related outputs 178 to one or more stability criteria 187. In some embodiments, the one or more stability criteria 187 include a threshold value, a rank cutoff, a percentile cutoff, a pass/fail rule, a staged screening rule, or combinations thereof. In some embodiments, candidate materials satisfying the one or more stability criteria 187 are passed to one or more downstream modules. In some embodiments, candidate materials not satisfying the one or more stability criteria 187 are removed, flagged for lower priority, or stored for possible later reconsideration.

In some embodiments, at step 1014, the recognition stability screening module 170 generates one or more filtered candidate subsets. In some embodiments, the one or more filtered candidate subsets include a stable-priority subset 188A, a borderline subset 188B, and a deprioritized subset 188C. In some embodiments, the stable-priority subset 188A is forwarded to the recognition-based property prediction module 150, the recognition composition optimization module 160, the recognition-based structure optimization module 140, or the combined scoring and ranking module 180. In some embodiments, the borderline subset 188B is retained for possible later analysis under revised thresholds or additional data conditions. In some embodiments, the deprioritized subset 188C is excluded from one or more downstream high-cost computational stages.

In some embodiments, the process of FIG. 8 is used to replace a binary stable-versus-unstable decision with a graded ranking policy. In some embodiments, candidate materials that would otherwise be treated identically under a hard threshold are instead distinguished by relative stability score. In some embodiments, this graded ranking policy improves candidate prioritization by allowing the system 100 to differentiate among candidates that are all nominally above or below a conventional cutoff.

In some embodiments, the process of FIG. 8 is applied to battery materials to evaluate electrochemical or thermodynamic stability. In some embodiments, the process of FIG. 8 is applied to semiconductors, metals, alloys, ceramics, polymers, catalytic materials, coating materials, two-dimensional materials, perovskites, and metal-organic frameworks. In some embodiments, the selected stability-associated metrics 184 differ across material classes while the same recognition coverage function 122 is used to determine the one or more recognition-weighted stability values 174.

In some embodiments, the process of FIG. 8 is carried out before property evaluation so that lower-stability candidates are removed before property prediction is performed. In some embodiments, the process of FIG. 8 is carried out after composition screening so that only composition-promising candidates are stability screened. In some embodiments, the process of FIG. 8 is carried out iteratively as new stability-related values become available from updated simulations, revised databases, or newly acquired experimental data.

In some embodiments, the one or more stability-related outputs 178 generated by the process of FIG. 8 are provided to the combined scoring and ranking module 180. In some embodiments, the one or more stability-related outputs 178 are also stored in the candidate-material records 332, the recognition metrics 336, or the ranking results 338. In some embodiments, the one or more stability-related outputs 178 are displayed through the user interface components 350 or transmitted through the communication interfaces 340.

In some embodiments, the pass-rate output represents a fraction of candidate materials satisfying one or more stability criteria 187 within a candidate set. In some embodiments, the pass-rate output is used to characterize the overall quality of a candidate pool, compare different candidate-generation strategies, tune one or more thresholds, or guide a user in selecting a downstream workflow configuration.

In some embodiments, the process of FIG. 8 provides a scalable and bounded stability-evaluation framework in which one or more stability-associated metrics 184 are processed through the recognition coverage function 122 to generate stability-related outputs 178 useful for ranking, filtering, and prioritization. In some embodiments, the one or more stability criteria 187 include at least one of: (i) an energy-above-hull cutoff selected in a range from about 0.01 eV/atom to about 0.20 eV/atom, (ii) a percentile cutoff selected in a range from about 1% to about 20% within a candidate set, or (iii) a top-K selection rule in which K is selected in a range from about 10 to about 10,000 candidates. In some embodiments, the process of FIG. 8 enables the system 100 to reduce downstream computational burden while maintaining a more informative decision structure than a simple binary threshold rule.

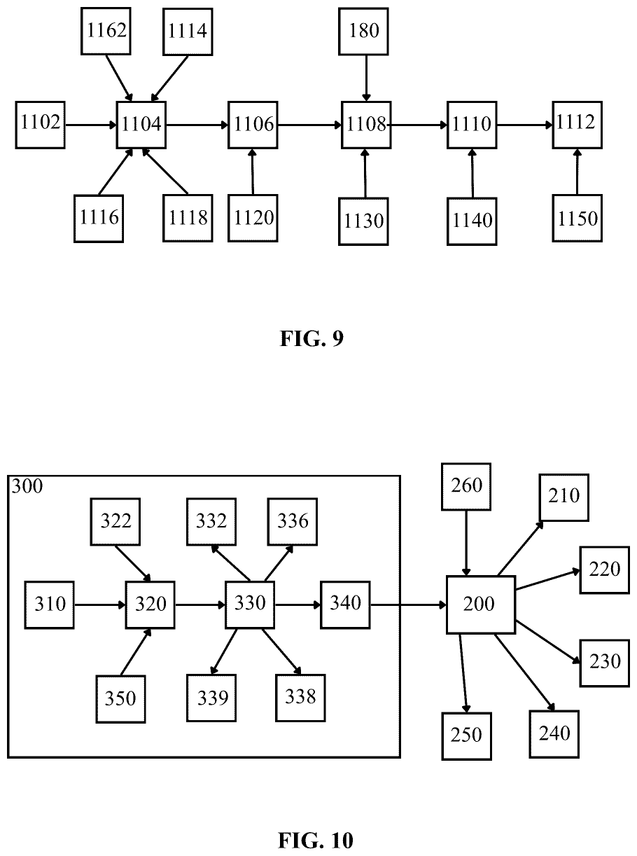

FIG. 9 illustrates an example combined candidate-material scoring process carried out by the combined scoring and ranking module 180. In some embodiments, the process of FIG. 9 is used to combine outputs from a plurality of recognition-enhanced computational subsystems so as to generate an overall candidate-material score, a candidate ranking, a shortlist, a prioritization output, or another downstream decision output.

In some embodiments, the process of FIG. 9 begins at step 1102, where the combined scoring and ranking module 180 receives one or more subsystem outputs 1104. In some embodiments, the one or more subsystem outputs 1104 include one or more structure-related outputs 148 from the recognition-based structure optimization module 140, one or more property-related outputs 158 from the recognition-based property prediction module 150, one or more composition-related outputs 168 from the recognition composition optimization module 160, and one or more stability-related outputs 178 from the recognition stability screening module 170. In some embodiments, fewer than all such subsystem outputs are used. In some embodiments, additional subsystem outputs derived from other recognition-enhanced analyses are also used.

In some embodiments, at step 1104, the combined scoring and ranking module 180 determines one or more component scores 1110 from the subsystem outputs 1104. In some embodiments, the one or more component scores 1110 include a structure-related score 1162, a property-related score 1114, a composition-related score 1116, and a stability-related score 1118. In some embodiments, one or more of the component scores 1110 are directly received from an upstream module. In some embodiments, one or more of the component scores 1110 are derived by rescaling, normalizing, threshold conditioning, or otherwise processing subsystem outputs before combination.

In some embodiments, the structure-related score 1162 is based on one or more structure scores, a structure ranking value, a recognition-weighted structure energy, a local-environment score, or combinations thereof. In some embodiments, the property-related score 1114 is based on a predicted property value, a property ranking score, a screening value, a thresholding value, a prioritization value, or combinations thereof. In some embodiments, the composition-related score 1116 is based on a composition score, a composition ranking value, a composition filter output, a preferred-composition subset, or combinations thereof. In some embodiments, the stability-related score 1118 is based on a stability score, a thresholding output, a filtered candidate subset, a pass-rate output, a stability ranking output, or combinations thereof.

In some embodiments, at step 1106, the combined scoring and ranking module 180 determines one or more combination weights 1120. In some embodiments, the one or more combination weights 1120 include a first weight associated with the property-related score 1114, a second weight associated with the stability-related score 1118, a third weight associated with the composition-related score 1116, and, in some embodiments, a fourth weight associated with the structure-related score 1162. In some embodiments, the one or more combination weights 1120 are fixed values. In some embodiments, the one or more combination weights 1120 are user-selected values. In some embodiments, the one or more combination weights 1120 are selected according to a material-class-specific policy, a workflow-stage-specific policy, an optimization-objective policy, or another configuration policy.

In some embodiments, the one or more combination weights 1120 are determined according to a parameter policy based on Xopt. In some embodiments, a normalization factor Z is defined as 1+Xopt+(Xopt squared). In some embodiments, a first weight is defined as 1/Z. In some embodiments, a second weight is defined as Xopt/Z. In some embodiments, a third weight is defined as (Xopt squared)/Z. In some embodiments, such a weighting policy provides a parameter-light combination framework that remains mathematically linked to the same recognition constant used in the recognition coverage function 122.

In some embodiments, at step 1108, the combined scoring and ranking module 180 determines an overall material score 1130 for a candidate material. In some embodiments, the overall material score 1130 is determined by combining the component scores 1110 according to the one or more combination weights 1120. In some embodiments, the overall material score 1130 is determined as a weighted sum of the property-related score 1114, the stability-related score 1118, and the composition-related score 1116. In some embodiments, the overall material score 1130 further includes the structure-related score 1162. In some embodiments, the overall material score 1130 is used as a basis for ranking candidate materials within a candidate set.

In some embodiments, the combined scoring and ranking module 180 uses a reduced-component embodiment. In some embodiments, only a property-related score and a stability-related score are combined. In some embodiments, only a property-related score, a stability-related score, and a composition-related score are combined. In some embodiments, the structure-related score 1162 is omitted when structural evaluation is unavailable, unnecessary, or deferred to a later workflow stage. In some embodiments, the combined scoring and ranking module 180 uses whichever component scores are available for a given candidate material while still producing an overall ranking or prioritization output.

In some embodiments, at step 1110, the combined scoring and ranking module 180 determines one or more ranking outputs 1140 from the one or more overall material scores 1130. In some embodiments, the one or more ranking outputs 1140 include a ranked candidate list, a top-subset output, a threshold-passing subset, a candidate-priority queue, or combinations thereof. In some embodiments, the ranked candidate list orders candidates from higher-priority to lower-priority. In some embodiments, the top-subset output identifies a selected number of higher-ranked candidates for downstream simulation or experimental validation.

In some embodiments, at step 1112, the combined scoring and ranking module 180 generates one or more downstream decision outputs 1150. In some embodiments, the one or more downstream decision outputs 1150 include a simulation-selection output, an experimental-validation output, a synthesis-selection output, a storage-or-archive output, a rejection output, or combinations thereof. In some embodiments, the simulation-selection output identifies one or more candidate materials to be passed to one or more external computational platforms 200 for higher-fidelity analysis. In some embodiments, the experimental-validation output identifies one or more candidate materials to be fabricated, synthesized, tested, or otherwise physically evaluated.

In some embodiments, the process of FIG. 9 uses a threshold-plus-ranking embodiment. In some embodiments, candidate materials are first ranked according to the overall material score 1130 and then filtered according to one or more threshold criteria. In some embodiments, candidate materials are first filtered according to one or more threshold criteria and then ranked. In some embodiments, the one or more threshold criteria are absolute score thresholds, percentile thresholds, top-K rules, diversity constraints, material-class-specific constraints, or combinations thereof.

In some embodiments, the process of FIG. 9 uses a multi-objective embodiment. In some embodiments, the combined scoring and ranking module 180 identifies one or more Pareto-efficient candidate materials using two or more of the component scores 1110. In some embodiments, the combined scoring and ranking module 180 combines weighted scoring with one or more Pareto-front analyses, diversity-selection rules, or constraint-aware shortlist policies. In some embodiments, such an embodiment is useful when a user seeks candidates that satisfy multiple partially competing objectives.

In some embodiments, the process of FIG. 9 is iterative. In some embodiments, one or more candidate materials selected using the overall material score 1130 are further simulated, experimentally evaluated, or otherwise refined, and new subsystem outputs are then returned to the combined scoring and ranking module 180. In some embodiments, this iterative process updates one or more component scores 1110, one or more combination weights 1120, or one or more downstream decision outputs 1150 over time.

In some embodiments, the overall material scores 1130, ranking outputs 1140, and downstream decision outputs 1150 are stored in the ranking results 338, candidate-material records 332, or other memory structures of the system 100. In some embodiments, such outputs are displayed through the user interface components 350, transmitted through the communication interfaces 340, or exported to one or more external computational platforms 200 or user devices.

In some embodiments, the process of FIG. 9 is applied to candidate materials from a single material class. In some embodiments, the process of FIG. 9 is applied to candidate materials from multiple different material classes within the same screening environment. In some embodiments, the same combined scoring and ranking framework is used to compare metals, alloys, semiconductors, ceramics, polymers, composites, battery materials, two-dimensional materials, perovskites, metal-organic frameworks, catalytic materials, coating materials, or combinations thereof, provided that suitable subsystem outputs are available.

In some embodiments, the process of FIG. 9 provides a unified decision layer for the system 100 by allowing outputs from structure analysis, property analysis, composition analysis, and stability analysis to be combined under a common recognition-enhanced scoring framework. In some embodiments, this unified decision layer reduces fragmentation across different screening stages and enables more coherent candidate prioritization across a complex computational materials-design workflow.

The foregoing description sets forth example system-level and module-level embodiments of the disclosed recognition-enhanced material design system 100. The following examples describe selected implementations and use cases in which the system 100, the recognition-enhanced computational framework 120, and one or more of the modules 140, 150, 160, 170, and 180 are applied to particular candidate-material evaluation tasks. The following examples are provided to illustrate operation of the disclosed technology and should not be construed as limiting the scope of the present disclosure.

In some embodiments, the examples described below use one or more normalized or otherwise recognition-compatible metrics derived from structural quantities, property quantities, composition quantities, stability quantities, or combinations thereof. In some embodiments, such examples illustrate how candidate materials may be ranked, filtered, or prioritized using the recognition coverage function 122 in conjunction with one or more baseline computational or data-derived inputs. In some embodiments, the examples also illustrate how the disclosed framework may be used as a post-processing layer on top of one or more external computational platforms 200.

In some embodiments, the examples described below are implemented using bulk crystalline materials. In some embodiments, the examples described below are implemented using alloy systems, semiconductor systems, battery systems, or two-dimensional materials systems. In some embodiments, one or more numerical values described below are illustrative of a selected embodiment and do not limit the disclosed framework to any particular material class, parameter range, or computational environment. For example, in a non-limiting illustrative calculation, if r=0.05 and Xopt=0.50, then Fcov(r)=0.05/(0.05+0.50)≈0.091; and if r=0.20 and Xopt=0.50, then Fcov(r)=0.20/(0.20+0.50)≈0.286, thereby producing a bounded weighting value that increases with increasing r.

In one example embodiment, the recognition-based structure optimization module 140 is used to evaluate a candidate crystalline structure using one or more pairwise interaction quantities and one or more corresponding distance-related quantities. In some embodiments, the candidate crystalline structure is represented by atomic positions, lattice parameters, and species assignments included within the structural inputs 142. In some embodiments, one or more pairwise distance values are determined between selected atoms or sites within the candidate crystalline structure.

In some embodiments, one or more of the pairwise distance values are normalized relative to a reference distance to generate one or more normalized distance metrics 143. In some embodiments, the reference distance is selected as a nearest-neighbor distance or another structural reference value associated with the candidate structure. In some embodiments, the normalized distance metrics 143 are then processed using the recognition coverage function 122 to generate one or more recognition coverage values associated with corresponding atom pairs or site pairs.

In some embodiments, one or more interaction-related quantities 145 are also determined for the corresponding atom pairs or site pairs. In some embodiments, the interaction-related quantities 145 are pairwise energy-related quantities. In some embodiments, the interaction-related quantities 145 are magnitude-based structural metrics. In some embodiments, the interaction-related quantities 145 are obtained from an external simulation engine. In some embodiments, the interaction-related quantities 145 are generated internally by the system 100.

In some embodiments, the recognition-based structure optimization module 140 determines one or more recognition-weighted pairwise values 144 by combining the one or more interaction-related quantities 145 with corresponding recognition coverage values. In some embodiments, a recognition-weighted pairwise value 144 is determined by multiplying a pairwise interaction-related quantity by a recognition coverage value associated with a corresponding normalized distance metric 143. In some embodiments, the recognition-weighted pairwise values 144 are aggregated across a plurality of pairs to generate a structure-related output 148, such as a structure score or a recognition-weighted structure energy.

In one illustrative implementation, a first candidate structure and a second candidate structure having the same nominal composition but different local arrangements are each evaluated using the recognition-based structure optimization module 140. In some embodiments, the first candidate structure produces a higher aggregated structure score than the second candidate structure. In some embodiments, the first candidate structure is therefore ranked above the second candidate structure and is selected for further simulation or structural refinement. In some embodiments, the second candidate structure is deprioritized or removed from a downstream workflow.

In some embodiments, the recognition-weighted pairwise values 144 are also used to identify one or more local regions of interest 720, such as defect-adjacent regions, under-coordinated regions, interface regions, or strained regions. In some embodiments, such local regions of interest 720 are then targeted for structural refinement, dopant relocation, interface modification, or other follow-on design operations.

In some embodiments, the example recognition-enhanced structure evaluation described above is carried out as a post-processing operation after a separate external engine has performed an initial relaxation. In some embodiments, the example recognition-enhanced structure evaluation is carried out iteratively, with updated coordinates or other structural values being reprocessed through the recognition-based structure optimization module 140 until one or more stopping criteria are satisfied.

In some embodiments, the example recognition-enhanced structure evaluation demonstrates how the recognition coverage function 122 may be applied to distance-related metrics so as to generate bounded recognition-weighted structural contributions for ranking, filtering, and optimization of candidate material structures.

In another example embodiment, the recognition-based property prediction module 150 is used to evaluate one or more candidate materials using one or more baseline property values 153 and one or more recognition metrics 157 associated with such baseline property values 153. In some embodiments, the baseline property values 153 are obtained from one or more first-principles simulations, one or more molecular dynamics simulations, one or more machine-learning models, one or more databases, one or more empirical models, or combinations thereof. In some embodiments, the baseline property values 153 correspond to one or more bandgap values, elastic modulus values, conductivity values, diffusivity values, refractive-index values, dielectric values, adsorption-related values, capacity values, hardness values, thermal-conductivity values, or other candidate-material properties.

In some embodiments, one or more recognition metrics 157 are generated for the baseline property values 153 using the preprocessing and normalization module 130. In some embodiments, a recognition metric 157 is based on a normalized property value, a ratio relative to a reference value, a magnitude-based transform, a cohesive-energy-related quantity, a density-related quantity, a structural descriptor, or another recognition-compatible metric associated with the property under consideration. In some embodiments, the recognition metrics 157 are nonnegative quantities suitable for evaluation using the recognition coverage function 122.

In some embodiments, the recognition-based property prediction module 150 applies the recognition coverage function 122 to the one or more recognition metrics 157 to generate one or more recognition coverage values 161. In some embodiments, the one or more recognition coverage values 161 are then combined with corresponding baseline property values 153 and, in some embodiments, one or more weighting factors 159 to determine one or more recognition-weighted property terms 154. In some embodiments, the one or more recognition-weighted property terms 154 are aggregated to generate one or more property-related outputs 158.

In one illustrative implementation, a first candidate semiconductor material and a second candidate semiconductor material are evaluated using one or more baseline bandgap values and one or more corresponding recognition metrics 157. In some embodiments, the first candidate semiconductor material produces a higher property ranking score than the second candidate semiconductor material after recognition weighting is applied. In some embodiments, the first candidate semiconductor material is therefore ranked above the second candidate semiconductor material for further simulation, fabrication, or validation. In some embodiments, the property-related outputs 158 are used primarily as ranking values while the baseline property values 153 remain separately reportable as original simulation or database outputs.

In some embodiments, the recognition-based property prediction module 150 is used in a multi-property embodiment in which a candidate material is evaluated with respect to two or more distinct properties. In some embodiments, a first recognition-weighted property term corresponds to a first property channel, a second recognition-weighted property term corresponds to a second property channel, and a third recognition-weighted property term corresponds to a third property channel. In some embodiments, the resulting property-related outputs 158 are used to identify candidates that satisfy a desired balance among multiple target properties rather than optimizing a single property in isolation.

In some embodiments, the recognition-based property prediction module 150 is used in a thresholding embodiment in which one or more property-related outputs 158 are compared to one or more property criteria. In some embodiments, candidate materials having property ranking scores or screening values above a threshold are passed to downstream stability screening, structural analysis, or combined ranking. In some embodiments, lower-ranked candidates are deprioritized before additional computational or experimental resources are committed.

In some embodiments, the example recognition-enhanced property evaluation described above demonstrates how one or more baseline property values 153 may be recognition weighted using the recognition coverage function 122 so as to produce property-related outputs 158 useful for screening, ranking, and prioritization of candidate materials across one or more material classes. In another example embodiment, the recognition composition optimization module 160 is used to evaluate one or more candidate material compositions based on one or more composition inputs 162 and one or more composition-associated metrics 163. In some embodiments, the composition inputs 162 include elemental identities, stoichiometric fractions, dopant definitions, host-species selections, site assignments, substitution candidates, alloy fractions, or combinations thereof. In some embodiments, the composition-associated metrics 163 include cohesive-energy-related values, compatibility-related values, valence-related values, size-mismatch-related values, electronegativity-related values, or other quantities relevant to composition ranking.

In some embodiments, the recognition composition optimization module 160 determines one or more normalized composition metrics 165 from the composition-associated metrics 163 and applies the recognition coverage function 122 to determine one or more recognition-weighted composition values 164. In some embodiments, the recognition-weighted composition values 164 are combined with one or more composition fractions 166 to determine one or more composition-related outputs 168, such as a composition score or a composition ranking value.

In one illustrative implementation, a plurality of candidate alloy compositions are evaluated using the recognition composition optimization module 160. In some embodiments, the candidate alloy compositions correspond to different combinations of metallic elements and different fractional compositions. In some embodiments, a first alloy composition produces a higher composition score than a second alloy composition. In some embodiments, the first alloy composition is therefore advanced to downstream structural evaluation or property analysis, whereas the second alloy composition is deprioritized or removed from the active candidate set.

In some embodiments, the recognition composition optimization module 160 is used to evaluate dopant choices for a host material. In some embodiments, a plurality of dopant identities or dopant fractions are compared using one or more recognition-weighted composition values 164 so as to identify a smaller subset of dopant candidates for downstream simulation or experimental testing. In some embodiments, the recognition composition optimization module 160 is used to reduce the size of a combinatorially large composition space before higher-cost analyses are performed. In some embodiments, one or more constraints 169 are also applied during the composition evaluation. In some embodiments, the one or more constraints 169 include charge-balance constraints, site-capacity constraints, manufacturability constraints, cost constraints, abundance constraints, or toxicity constraints. In some embodiments, a candidate composition having a favorable composition score may still be excluded if one or more applicable constraints 169 are not satisfied.

In some embodiments, the example recognition-enhanced composition evaluation described above demonstrates how the recognition coverage function 122 may be used to generate bounded composition-related outputs 168 useful for ranking and filtering candidate compositions across a broad search space.

In another example embodiment, the recognition stability screening module 170 is used to evaluate one or more candidate materials using one or more stability inputs 172. In some embodiments, the one or more stability inputs 172 include formation-enthalpy-related values, energy-above-hull values, decomposition-energy values, free-energy values, phase-stability values, metastability values, or combinations thereof. In some embodiments, one or more stability-associated metrics 184 are derived from the one or more stability inputs 172 and are converted into nonnegative quantities suitable for recognition weighting.

In some embodiments, the recognition stability screening module 170 generates one or more normalized stability metrics 185 and applies the recognition coverage function 122 to determine one or more recognition-weighted stability values 174. In some embodiments, the one or more recognition-weighted stability values 174 are used to determine one or more stability-related outputs 178, including a stability score, a thresholding output, a filtered candidate subset, a pass-rate output, or a stability ranking output.

In one illustrative implementation, a first candidate material, a second candidate material, and a third candidate material are each evaluated using one or more formation-enthalpy-related values or energy-above-hull values. In some embodiments, the first candidate material produces a higher stability score than the second candidate material, and the second candidate material produces a higher stability score than the third candidate material. In some embodiments, the first candidate material is passed directly to downstream property evaluation, the second candidate material is retained in a borderline subset 188B, and the third candidate material is assigned to the deprioritized subset 188C.

In some embodiments, the recognition stability screening module 170 therefore replaces a hard binary stable-versus-unstable rule with a graded ranking approach. In some embodiments, this graded ranking approach allows candidates having similar conventional stability classifications to be differentiated and prioritized more effectively for downstream analysis.

In some embodiments, the example recognition-enhanced stability evaluation described above demonstrates how one or more stability-associated metrics 184 may be processed using the recognition coverage function 122 to produce stability-related outputs 178 useful for ranking, filtering, and candidate-pool reduction.

In another example embodiment, outputs from a plurality of modules are combined using the combined scoring and ranking module 180. In some embodiments, the combined scoring and ranking module 180 receives one or more property-related outputs 158, one or more composition-related outputs 168, one or more stability-related outputs 178, and, in some embodiments, one or more structure-related outputs 148. In some embodiments, the combined scoring and ranking module 180 determines one or more component scores 1110 and combines such component scores 1110 using one or more combination weights 1120 to determine an overall material score 1130.

In one illustrative implementation, a candidate battery material is evaluated using a property-related score 1114 associated with one or more conductivity-related or capacity-related values, a stability-related score 1118 associated with one or more stability metrics, and a composition-related score 1116 associated with one or more host or dopant selections. In some embodiments, the combined scoring and ranking module 180 determines an overall material score 1130 from such component scores. In some embodiments, the candidate battery material is then compared against one or more additional battery-material candidates using the overall material score 1130.

In some embodiments, the combined scoring and ranking module 180 generates a ranked candidate list and a top-subset output from a plurality of overall material scores 1130. In some embodiments, the top-subset output is used to identify a smaller set of candidates for higher-fidelity simulation, fabrication, synthesis, or experimental validation. In some embodiments, the remaining candidates are stored for later reconsideration, archived, or rejected.

In some embodiments, the combined scoring and ranking module 180 is used in a multi-objective embodiment in which a desired property level, a desired stability level, and a desired composition preference are all considered together. In some embodiments, the combined scoring and ranking module 180 thereby provides a unified decision layer through which candidates from a large initial set are converted into a smaller and more actionable shortlist.

In some embodiments, the example combined candidate-material scoring described above demonstrates how outputs from different recognition-enhanced modules may be integrated into a common ranking framework so that candidate materials can be compared, filtered, and prioritized using a coordinated and bounded scoring policy.

In another example embodiment, the disclosed recognition-enhanced computational framework 120 is used in a high-throughput screening environment. In some embodiments, an initial candidate set includes a large plurality of candidate materials generated by enumeration, database retrieval, machine-learning proposal, or another candidate-generation process. In some embodiments, the initial candidate set is processed through one or more of the recognition stability screening module 170, the recognition composition optimization module 160, the recognition-based property prediction module 150, and the recognition-based structure optimization module 140 in a staged workflow.

In some embodiments, a first subset of candidate materials is removed after stability screening, a second subset is removed after composition evaluation, and a third subset is removed after property evaluation, thereby leaving a smaller finalist set for structure optimization, higher-fidelity simulation, or experimental validation. In some embodiments, the disclosed framework reduces downstream computational burden by applying bounded recognition-weighted ranking and filtering operations before the most expensive stages of analysis are performed.

In some embodiments, the high-throughput screening environment is applied to semiconductors, battery materials, high-entropy alloys, two-dimensional materials, catalytic materials, coating materials, or other candidate-material classes. In some embodiments, the same recognition coverage function 122 is used across multiple stages of such high-throughput screening, thereby providing a common ranking logic across otherwise different computational tasks.

In some embodiments, the foregoing examples demonstrate that the disclosed system 100 may be used as a flexible recognition-enhanced framework for evaluating candidate materials across multiple analysis domains, multiple workflow stages, and multiple material classes. In some embodiments, the disclosed framework enables bounded and coordinated ranking, filtering, prediction, and prioritization while remaining compatible with one or more external computational platforms 200 and one or more local, distributed, or cloud-based computing environments 300. It will be appreciated that the disclosed recognition-enhanced material design system 100 may be implemented in a variety of forms and configurations without departing from the scope of the present disclosure. In some embodiments, the system 100 is implemented as a standalone software application executed on a local workstation. In some embodiments, the system 100 is implemented as a distributed workflow executed across a plurality of networked computational resources. In some embodiments, the system 100 is implemented as a cloud-based service, an application programming interface, a plugin, a wrapper framework, a batch-processing environment, or another computational deployment architecture.

In some embodiments, the system 100 is configured to interact with one or more user-defined workflow settings. In some embodiments, such workflow settings include candidate-set size, screening thresholds, normalization policies, ranking policies, aggregation rules, material-class selections, property targets, stability criteria, composition constraints, optimization criteria, or combinations thereof. In some embodiments, one or more workflow settings are stored in the configuration data 339 and are selectively applied during execution of the preprocessing and normalization module 130, the recognition-based structure optimization module 140, the recognition-based property prediction module 150, the recognition composition optimization module 160, the recognition stability screening module 170, or the combined scoring and ranking module 180.

In some embodiments, one or more modules of the system 100 are omitted. In some embodiments, additional modules are added. In some embodiments, the recognition-based structure optimization module 140 is omitted in workflows focused primarily on composition and property screening. In some embodiments, the recognition composition optimization module 160 is omitted in workflows in which candidate compositions are fixed in advance. In some embodiments, the recognition stability screening module 170 is omitted when stability information is unavailable or deferred to a later stage. In some embodiments, the combined scoring and ranking module 180 operates using whichever subsystem outputs are available for a particular candidate material.

In some embodiments, the disclosed framework is applied to a single candidate material. In some embodiments, the disclosed framework is applied to a plurality of candidate materials within a candidate set. In some embodiments, the disclosed framework is applied to a dynamically changing candidate set in which new candidates are added, removed, modified, or re-ranked over time. In some embodiments, candidate materials are grouped by material class, target application, source dataset, computational provenance, or another organizational criterion prior to or during recognition-enhanced evaluation.

In some embodiments, one or more outputs generated by the system 100 are stored together with provenance data indicating one or more source engines, one or more input datasets, one or more normalization settings, one or more ranking settings, one or more threshold settings, one or more timestamps, or one or more workflow identifiers. In some embodiments, such provenance data supports reproducibility, auditing, later comparison of candidate-evaluation runs, or subsequent retraining or recalibration of one or more upstream predictive models.

In some embodiments, the disclosed framework is used in conjunction with one or more fabrication workflows, laboratory workflows, qualification workflows, procurement workflows, or deployment workflows. In some embodiments, a ranked subset of candidate materials generated by the combined scoring and ranking module 180 is supplied to a downstream synthesis team, test platform, fabrication process, or qualification environment. In some embodiments, feedback from such downstream activities is returned to the system 100 and stored in the candidate-material records 332 for further recognition-enhanced analysis.

In some embodiments, the disclosed framework is used with one or more human-in-the-loop decision processes. In some embodiments, a user reviews one or more candidate rankings 192, screening decisions 194, prediction outputs 196, or optimization outputs 198 before selecting a downstream action. In some embodiments, one or more downstream actions are executed automatically according to predefined workflow rules. In some embodiments, the system 100 supports a hybrid workflow in which some actions are automated and other actions are subject to analyst approval.

In some embodiments, the disclosed framework is compatible with a wide range of computational fidelities. In some embodiments, low-cost baseline property values 153 or stability inputs 172 are used in early-stage screening, while higher-fidelity values are introduced in later-stage evaluation. In some embodiments, lower-fidelity and higher-fidelity values are both processed within the same overall candidate-ranking framework. In some embodiments, the system 100 therefore supports staged discovery pipelines in which computational effort is increased progressively as the candidate pool is reduced.

In some embodiments, the disclosed framework is not limited to any specific software language, data structure, database format, operating system, hardware platform, network topology, or machine-learning architecture. In some embodiments, program instructions 322 implementing the disclosed operations are written in one or more compiled languages, interpreted languages, scripting languages, or combinations thereof. In some embodiments, the disclosed operations are embodied in software, firmware, hardware logic, or combinations thereof.

In some embodiments, the one or more processors 310 execute program instructions 322 stored in the one or more memory devices 320 to cause the system 100 to perform the operations described herein. In some embodiments, one or more of such operations are distributed across multiple processors, multiple memory devices, multiple servers, multiple cluster nodes, or multiple cloud instances. In some embodiments, one or more operations are accelerated using graphics processing units, tensor processing units, field-programmable gate arrays, or other specialized computational resources.

Unless otherwise expressly stated, no particular order of operations is required for the methods described herein. In some embodiments, steps described in connection with one figure are performed in an order different from that expressly illustrated or described. In some embodiments, two or more steps are performed concurrently. In some embodiments, one or more steps are repeated, omitted, subdivided, merged, or otherwise modified according to a selected implementation.

As used herein, relational terms such as first, second, third, and the like are used solely to distinguish one entity or action from another and do not necessarily require or imply any actual such relationship or order between such entities or actions. The terms comprising, including, having, containing, and involving are intended to be open-ended terms that specify the presence of stated features, integers, steps, operations, elements, or components, but do not preclude the presence or addition of one or more other features, integers, steps, operations, elements, components, or groups thereof.

As used herein, the singular forms a, an, and the include plural referents unless the context clearly indicates otherwise. As used herein, the term or is intended to be inclusive unless the context clearly indicates otherwise. Numeric ranges described herein are intended to include the endpoints of such ranges and all intermediate values and subranges unless the context clearly indicates otherwise. Although certain embodiments have been described herein in connection with particular examples, implementations, and figures, the disclosed subject matter is not limited to those specific embodiments. Various modifications, substitutions, changes, and equivalents will be apparent to persons of ordinary skill in the art in view of the present disclosure. Accordingly, the scope of the present disclosure is defined by the appended claims and their equivalents.

What is claimed is:

1. A computer-implemented method for evaluating candidate materials, the method comprising:
receiving, by one or more processors, candidate-material inputs associated with a candidate material;
generating, from the candidate-material inputs, one or more recognition-compatible metrics;
applying a recognition coverage function to at least one of the one or more recognition-compatible metrics, wherein the recognition coverage function is defined as Fcov(r)=r/(r+Xopt), wherein r is a nonnegative input metric and Xopt is a positive recognition constant; generating, using the recognition coverage function, one or more recognition-weighted outputs associated with the candidate material; and determining, from the one or more recognition-weighted outputs, at least one of a ranking output, a screening output, a prediction output, an optimization output, or a prioritization output for the candidate material, wherein determining the at least one of the ranking output, the screening output, the prediction output, the optimization output, or the prioritization output comprises generating a ranked candidate list and selecting, based on at least one of (i) a threshold value, (ii) a rank cutoff, (iii) a percentile cutoff, or (iv) a top-K criterion, a subset of candidate materials for at least one of further simulation, experimental validation, synthesis, fabrication, or qualification.

2. The method of claim 1, wherein generating the one or more recognition-weighted outputs comprises generating one or more recognition-weighted structural outputs from structural information associated with the candidate material.

3. The method of claim 2, wherein generating the one or more recognition-weighted structural outputs comprises:
determining one or more distance-related metrics associated with pairs of structural entities of the candidate material;
generating one or more normalized distance metrics from the one or more distance-related metrics;
applying the recognition coverage function to the one or more normalized distance metrics to generate one or more recognition coverage values;
combining the one or more recognition coverage values with one or more interaction-related quantities associated with the pairs of structural entities to generate one or more recognition-weighted pairwise values; and
aggregating the one or more recognition-weighted pairwise values to generate a structure-related output, wherein the structure-related output comprises at least one of a structure score, a structure ranking value, a recognition-weighted structure energy, a local environment score, or a relaxed structure output.

4. A non-transitory computer-readable medium storing instructions that, when executed by one or more processors, cause the one or more processors to: receive candidate-material inputs associated with a candidate material; generate one or more recognition-compatible metrics from the candidate-material inputs; apply a recognition coverage function to at least one of the one or more recognition-compatible metrics, wherein the recognition coverage function is defined as Fcov(r)=r/(r+Xopt), wherein r is a nonnegative input metric and Xopt is a positive recognition constant; generate, using the recognition coverage function, one or more recognition-weighted outputs associated with the candidate material; and determine, from the one or more recognition-weighted outputs, at least one of a ranking output, a screening output, a prediction output, an optimization output, or a prioritization output for the candidate material, wherein determining the at least one of the ranking output, the screening output, the prediction output, the optimization output, or the prioritization output comprises generating a ranked candidate list and selecting, based on at least one of (i) a threshold value, (ii) a rank cutoff, (iii) a percentile cutoff, or (iv) a top-K criterion, a subset of candidate materials for at least one of further simulation, experimental validation, synthesis, fabrication, or qualification.

5. The method of claim 3, wherein the one or more interaction-related quantities comprise signed interaction-related quantities, and wherein generating the one or more recognition-weighted pairwise values comprises preserving a sign of a signed interaction-related quantity while recognition weighting a magnitude of the signed interaction-related quantity.

6. The method of claim 1, wherein generating the one or more recognition-weighted outputs comprises generating one or more recognition-weighted property outputs from one or more baseline property values associated with the candidate material.

7. The method of claim 6, wherein generating the one or more recognition-weighted property outputs comprises:
receiving one or more baseline property values and one or more recognition metrics associated with the one or more baseline property values;
applying the recognition coverage function to the one or more recognition metrics to generate one or more recognition coverage values;
combining the one or more baseline property values and the one or more recognition coverage values to generate one or more recognition-weighted property terms; and
aggregating the one or more recognition-weighted property terms to generate at least one of a predicted property value, a property ranking score, a screening value, a thresholding value, or a prioritization value.

8. The method of claim 7, wherein the one or more baseline property values comprise at least one of a bandgap value, an elastic modulus value, a conductivity value, a diffusivity value, a dielectric value, a refractive-index value, a hardness value, a thermal-conductivity value, a capacity value, or an adsorption-related value.

9. The method of claim 1, wherein generating the one or more recognition-weighted outputs comprises generating one or more recognition-weighted composition outputs from composition information associated with the candidate material.

10. The method of claim 9, wherein generating the one or more recognition-weighted composition outputs comprises:
receiving one or more composition inputs comprising elemental identities and composition fractions;
determining one or more composition-associated metrics from the one or more composition inputs;
generating one or more normalized composition metrics from the one or more composition-associated metrics;
applying the recognition coverage function to the one or more normalized composition metrics to generate one or more recognition-weighted composition values; and
combining the one or more recognition-weighted composition values with the composition fractions to generate at least one of a composition score, a composition ranking value, a composition filter output, or a preferred-composition subset.

11. The method of claim 10, wherein the one or more composition-associated metrics comprise at least one of a cohesive-energy-related quantity, a compatibility-related quantity, a valence-related quantity, a size-mismatch-related quantity, an electronegativity-related quantity, or a site-preference-related quantity.

12. The method of claim 1, wherein generating the one or more recognition-weighted outputs comprises generating one or more recognition-weighted stability outputs from one or more stability-related inputs associated with the candidate material.

13. The method of claim 12, wherein generating the one or more recognition-weighted stability outputs comprises:
receiving one or more stability inputs;
determining one or more nonnegative stability-associated metrics from the one or more stability inputs;
applying the recognition coverage function to the one or more nonnegative stability-associated metrics to generate one or more recognition-weighted stability values; and
determining, from the one or more recognition-weighted stability values, at least one of a stability score, a thresholding output, a filtered candidate subset, a pass-rate output, or a stability ranking output.

14. The method of claim 13, wherein the one or more stability inputs comprise at least one of a formation-enthalpy-related value, an energy-above-hull value, a decomposition-energy value, a free-energy value, a phase-stability value, or a metastability value.

15. The method of claim 1, further comprising combining two or more of a structure-related output, a property-related output, a composition-related output, and a stability-related output to determine an overall material score for the candidate material.

16. The method of claim 15, wherein determining the overall material score comprises combining a property-related score, a stability-related score, and a composition-related score according to one or more weights.

17. The method of claim 16, wherein the one or more weights are user-selected weights, fixed weights, or weights determined according to a parameter policy based on Xopt.

18. The method of claim 1, further comprising interfacing with one or more external computational platforms to receive at least a portion of the candidate-material inputs, wherein the one or more external computational platforms comprise at least one of a first-principles simulation platform, a molecular dynamics platform, a machine-learning platform, or a database platform, wherein the one or more recognition-weighted outputs are generated without modifying source code of the one or more external computational platforms, and wherein determining the at least one of the ranking output, the screening output, the prediction output, the optimization output, or the prioritization output comprises generating a ranked candidate list and selecting a subset of candidate materials for at least one of further simulation, experimental validation, synthesis, fabrication, or qualification.

19. The method of claim 1, wherein Xopt equals phi divided by pi, and wherein phi equals the quantity one plus the square root of five, divided by two.

20. A system for evaluating candidate materials, the system comprising: one or more processors; one or more memory devices storing instructions that, when executed by the one or more processors, cause the one or more processors to implement: a preprocessing and normalization module configured to generate one or more recognition-compatible metrics from candidate-material inputs associated with a candidate material; a recognition-based structure optimization module configured to generate one or more recognition-weighted structural outputs; a recognition-based property prediction module configured to generate one or more recognition-weighted property outputs; a recognition composition optimization module configured to generate one or more recognition-weighted composition outputs; a recognition stability screening module configured to generate one or more recognition-weighted stability outputs; and a combined scoring and ranking module configured to determine, from one or more of the recognition-weighted outputs, at least one of a ranking output, a screening output, a prediction output, an optimization output, or a prioritization output for the candidate material, wherein the combined scoring and ranking module is further configured to generate a ranked candidate list and select, based on at least one of (i) a threshold value, (ii) a rank cutoff, (iii) a percentile cutoff, or (iv) a top-K criterion, a subset of candidate materials for at least one of further simulation, experimental validation, synthesis, fabrication, or qualification, wherein the one or more processors are further caused to apply a recognition coverage function to at least one recognition-compatible metric, wherein the recognition coverage function is defined as Fcov(r)=r/(r+Xopt), wherein r is a nonnegative input metric and Xopt is a positive recognition constant.
